SEQUENCE LISTING (9 sequences)

SEQ ID NO: 1 - protein, 17 aa, Artificial Sequence
  Antimicrobial peptide
  VARIANT 2, 12: Xaa = any amino acid
GXKRLVQRLKDXLRNLV

SEQ ID NO: 2 - protein, 17 aa, Artificial Sequence
  Antimicrobial peptide
  VARIANT 2, 12: Xaa = biphenylalanine
GXKRLVQRLKDXLRNLV

SEQ ID NO: 3 - protein, 8 aa, Artificial Sequence
  Antimicrobial peptide
  VARIANT (4)...(7): Xaa = any amino acid
WWWXXXXW

SEQ ID NO: 4 - protein, 6 aa, Artificial Sequence
  Antimicrobial peptide
RRRWWW

SEQ ID NO: 5 - protein, 7 aa, Artificial Sequence
  Antimicrobial peptide
RRRWWWW

SEQ ID NO: 6 - protein, 8 aa, Artificial Sequence
  Antimicrobial peptide
  VARIANT 8: Xaa = any amino acid
RRRWWWWX

SEQ ID NO: 7 - protein, 8 aa, Artificial Sequence
  Antimicrobial peptide
RRRWWWWV

SEQ ID NO: 8 - protein, 8 aa, Artificial Sequence
  Antimicrobial peptide
  VARIANT 1-3, 8: Xaa = any amino acid
XXXWWWWX

SEQ ID NO: 9 - protein, 20 aa, Artificial Sequence
  BMP-2 peptide
KIPKASSVPTELSAISTLYL

(19) United States
(12) Patent Application Publication
(10) Pub. No.: US 2022/0096706 A1
(43) Pub. Date: Mar. 31, 2022

(54) ELECTROSPUN NANOFIBER-BASED DRESSINGS AND METHODS OF MANUFACTURE AND USE THEREOF

(71) Applicant: BOARD OF REGENTS OF THE UNIVERSITY OF NEBRASKA, Lincoln, NE (US)

(72) Inventors: Jingwei XIE, Omaha, NE (US); Guangshun WANG, Omaha, NE (US)

(21) Appl. No.: 17/425,057

(22) PCT Filed: Jan. 28, 2020
(86) PCT No.: PCT/US2020/015349

§ 371 Date: Jul. 22, 2021

Related U.S. Application Data
(60) Provisional application No. 62/797,697, filed on Jan. 28, 2019.

(51) Int. Cl. A61L 15/44; D01D 5/00; D01F 8/14; D01F 8/18; A61L 15/32; A61L 15/22; A61L 15/26

(52) U.S. Cl. CPC A61L 15/44; D01D 5/0023; D01F 8/14; B82Y 5/00; A61L 15/32; A61L 15/225; A61L 15/26; D01F 8/18

(57) ABSTRACT

Nanofiber structures are provided as well as methods of use thereof and methods of making.

This application claims priority under 35 U.S.C. § 119(e) to U.S. Provisional Patent Application No. 62/797,697, filed Jan. 28, 2019. The foregoing application is incorporated by reference herein.

This invention was made with government support under Grant Nos. R01 AI105147, P20 GM103480, and R01 GM123081 awarded by the National Institutes of Health. The government has certain rights in the invention.

### FIELD OF THE INVENTION

This application relates to the fields of nanofiber structures. More specifically, this invention provides methods of synthesizing nanofiber structures and methods of use thereof.

### BACKGROUND OF THE INVENTION

Several publications and patent documents are cited throughout the specification in order to describe the state of the art to which this invention pertains. Each of these citations is incorporated herein by reference as though set forth in full.

Burn, trauma, surgery and chronic diseases induced wound infections caused by multi-drug resistant pathogens pose a major challenge to wound management (Branski, et al. (2009) Surg. Infect., 10(5):389-397; Wang, et al. (2018) Adv. Drug Deliv. Rev., 123:3-17). For example, diabetes mellitus affects 23.6 million people in the United States and approximately 20-25% of diabetic patients will develop foot ulceration during the course of their disease (Wukich, et al. (2010) J. Bone Joint Surg. Am., 92(2):287-295). Among them, 63.4% of diabetic patients develop multidrug-resistant organism infections (Lavery, et al. (2009) Diabetes Res. Clin. Pract., 83(3):347-352). Failure to prevent or manage such infections has resulted in amputation, sepsis, and even death (Sen, et al. (2009) Wound Repair Regen., 17(6):763-771). There is an urgent need for novel approaches for treating wound infections, particularly those caused by multi-drug resistant pathogens.

Current approaches to the treatment of wound infections mainly include the use of antibiotics and silver and surgical management (Kalan, et al. (2017) Int. J. Antimicrob. Agents, 49(6):719-726; Hingorani, et al. (2016) J. Vasc. Surg., 63(2):3S-21S; Abbas, et al. (2015) Expert Opin. Pharmaco., 16(6):821-832). In short, the use of antibiotics is a primary and foundational tool in the care of the injured personnel (Das, et al. (2017) Mol. Pharm., 14(6):1988-1997). Current evidence strongly suggests that this routine use of antibiotics may be an important factor in selecting resistant bacterial strains (Du, et al. (2015) Mol. Pharm., 12(5):1544-1553). Meanwhile, recent findings indicated some antibiotic actually delays the wound-healing process and that silver may have serious cytotoxic activity on various host cells, as well as potential side effects such as argyria, a medically benign but permanent bluish-gray discoloration of the skin (Bourdillon, et al. (2017) Int. Wound J 14(6):1066-1075; Atiyeh, et al. (2007) Burns, 33(2):139-148; Kwon, et al. (2009) Ann. Dermatol., 21(3):308-310). Furthermore, while antibiotic development continues to stagnate, antibiotic resistance continues to spread, particularly Gram-negative bacilli (Dik, et al. (2018) Chem. Rev., 118(12):5952-5984). Additionally, current antibiotic biomaterial formulations suffer from limitations including non-degradability (e.g., poly(methyl methacrylate) (PMMA)) or too short duration of release (e.g., collagen sponge) (Tong, et al. (2016) Wound Repair Regen., 24(1):45-56; Shi, et al. (2010) Biomaterials, 31(14):4146-4156). Accordingly, new means for treating wound infections are needed.

### SUMMARY OF THE INVENTION

In accordance with the instant invention, nanofiber structures, particularly antimicrobial loaded nanofiber structures, are provided. In a particular embodiment, the nanofiber structure comprises electrospun nanofibers having a core-shell morphology. The nanofibers may comprise an amphiphilic block copolymer (e.g., a poloxamer such as poloxamer 407), a hydrophobic polymer (e.g., polycaprolactone (PCL)), and one or more antimicrobials (e.g., antibiotic, antifungal, an antimicrobial peptide, silver nanoparticles, silver nitrate, gallium nitrate), wherein the antimicrobial is contained within the core of the nanofibers and/or the hydrophobic polymer is contained within the shell of the nanofibers. The amphiphilic block copolymer may be present at the interface of the core and shell of the nanofiber such that the hydrophilic domain is present in or interacts with the core and the hydrophobic domain is present in or interacts with the shell. The nanofibers of the nanofiber structure may be uniaxially-aligned nanofibers, random nanofibers, and/or entangled nanofibers. In a particular embodiment, the nanofiber structure is a porous nanofiber sponge or aerogel. The nanofibers of the nanofiber structure may further comprise a coating such as a coating comprising an antimicrobial (e.g., the same as or different than the antimicrobial in the core of the nanofibers) and/or an amphiphilic block copolymer (e.g., the same as or different than the an amphiphilic block copolymer the nanofibers). The nanofibers of the nanofiber structure may further comprise another drug or therapeutic agent such as a growth factor, a signaling molecule, a cytokine, or a hemostatic agent.

The nanofiber structures of the instant invention may also further comprise microneedles. For example, the nanofiber structure may be attached or fused to a plurality of microneedles or a microneedle structure comprising a base and a plurality of microneedles. The microneedles and/or microneedle structure may comprise an antimicrobial (e.g., the same as or different than the antimicrobial in the nanofibers) and a dissolvable polymer (e.g., polyvinylpyrrolidone (PVP) or polyvinyl alcohol (PVA)) or a biodegradable polymer (e.g., poly(lactic-co-glycolic acid) (PLGA)).

In accordance with another aspect of the instant invention, methods for producing a nanofiber structure of the instant invention are also provided.

In accordance with another aspect of the instant invention, wound dressings comprising a nanofiber structure of the instant invention are also provided.

In accordance with another aspect of the instant invention, methods of using the nanofiber structures are provided. For example, the nanofiber structures may be used to enhance and/or improve wound healing; reduce, inhibit, prevent, and/or eliminate an infection (e.g., a bacterial infection, particularly a bacterial biofilm); and/or inhibit bleeding.

### BRIEF DESCRIPTION OF THE DRAWINGS

FIG. 1A provides a schematic illustrating co-axial electrospinning and preparation of F127/17BIPHE2-PCL nanofiber dressings. FIG. 1B provides a schematic illustrating electrospraying deposition of molecularly engineered peptide 17BIPHE2 to F127/17BIPHE2-PCL nanofiber membranes to form F127/17BIPHE2-PCL-S nanofiber dressings.

FIG. 2A provides a macrograph of F127/17BIPHE2-PCL core-shell nanofiber membrane. FIGS. 2B-2D provide scanning electron microscopy (SEM) images of F127/PCL core-shell nanofibers, F127/17BIPHE2-PCL core-shell nanofibers, and F127/17BIPHE2-PCL-S nanofibers, respectively. FIG. 2E provides a laser confocal scanning microscopy (LCSM) image of F127/FITC-BSA-PCL core-shell nanofibers.

FIG. 3 provides in vitro release profiles of 17BIPHE2 peptides from F127/17BIPHE2-PCL core-shell nanofibers and after electrospraying deposition of 17BIPHE2 (F127/17BIPHE2-PCL-S).

FIG. 4 provides a graph of the in vitro antibacterial efficacy test of F127/17BIPHE2-PCL core-shell nanofibers. The bacteria solution was diluted into 1.0×10⁷ CFU/mL in PBS. One mg of PCL or F127/17BIPHE2-PCL core-shell nanofiber membranes was co-incubated with the bacteria solution for 2 hours at 37° C. Total living bacteria were determined by culturing on agar plates.

FIGS. 5A and 5B provide in vitro cytotoxicity test of 17BIPHE2 peptide-loaded PCL nanofiber membranes. FIG. 5A: alamarBlue® cell viability test against HaCat cells. FIG. 5B: alamarBlue® cell viability test against U937 cells. Each data point represents arithmetic mean±SD values from four samples.

FIG. 6A shows MRSA USA300 biofilm formation in chronic wounds based on type II diabetic mice. Wounds were created and fixed with splint, and MRSA was inoculated for 24 hours. FIG. 6B provides LIVE/DEAD® staining for the tissue collected from wounds after 24 hours of MRSA inoculation and subsequent 24 hour mupirocin 2% treatment. FIG. 6C provides SEM observation of the tissue collected from wounds after 24 hour of MRSA inoculation and subsequent 24 hour mupirocin 2% treatment. FIG. 6D provides quantification of bacterial load in the wound after 24 hours of MRSA inoculation and subsequent 24 hour and 48 hour mupirocin 2% treatment. FIG. 6E provides an image of a MRSA inhibition zone test of commercial antibiotic mupirocin 2%.

FIGS. 7A-7D show the in vivo antibiofilm efficacy test of 17BIPHE2/F127-PCL-S nanofiber dressings. The MRSA and P. aeruginosa biofilm-containing chronic wounds created in type II diabetic mice were treated by 17BIPHE2/F127-PCL-S nanofiber dressings without and with debridement. FIG. 7A: 3 days 1 change 17BIPHE2 against MRSA: the wounds were treated by 17BIPHE2/F127-PCL-S nanofiber dressings once for 3 days. FIG. 7B: 3 days 3 changes 17BIPHE2 against MRSA: the wounds were treated by 17BIPHE2/F127-PCL-S nanofiber dressings three times for 3 days with daily replacement. FIG. 7C: 7 days 7 changes 17BIPHE2: the wounds were treated by 17BIPHE2/F127-PCL-S nanofiber dressings seven times for 7 days with daily replacement. FIG. 7D: 3 days 3 changes 17BIPHE2 against P. aeruginosa: the wounds were treated by 17BIPHE2/F127-PCL-S nanofiber dressings three times for 3 days with daily replacement. p<0.05 marked as an asterisk (*).

FIGS. 8A-8D provide H&E staining for the tissue collected from biofilm-containing wounds treated by F127-PCL nanofiber dressings with once-daily dressing change for 7 days (FIG. 8A), F127-PCL nanofiber dressings in combination with debridement with once-daily dressing change for 7 days (FIG. 8B), F127/17BIPHE2-PCL nanofiber dressings with once-daily dressing change for 7 days (FIG. 8C), and F127/17BIPHE2-PCL nanofiber dressings in combination with debridement with once-daily dressing change for 7 days (FIG. 8D).

FIG. 9 provides graphs of the quantification of cytokines in wound tissue samples. Cytokines levels were measured on day 3 (closed bars) and day 7 (open bars). The experiment performed using the standard protocol provided by the manufacturer (LEGENDplex™ Mouse Inflammation Panel (13-plex) with Filter Plate). p<0.05 marked as *.

FIG. 10 provides a graph of the ex vivo antibacterial efficacy of F127/17BIPHE2-PCL core-shell nanofibers. P. aeruginosa (200 CFU/mL) was inoculated to the artificial wounds created on the human skin tissues. The wounds were covered by F127-PCL and F127/17BIPHE2-PCL-S nanofiber dressings for 2 and 8 hours. Bacterial burden was quantified based on the tissue lysis solution.

FIG. 11A provides a schematic illustrating Janus-type antimicrobial dressings comprising molecularly engineered peptides-loaded electrospun nanofiber membranes and microneedle arrays, e.g., for the treatment of biofilms in chronic wounds. FIG. 11B provides a schematic illustrating co-axial electrospinning and preparation of Pluronic® F127/W379-PCL nanofiber dressings and a schematic illustrating electrospray deposition of engineered peptide W379 to Pluronic® F127/W379-PCL nanofiber membranes to form Pluronic® F127/W379-PCL-S nanofiber dressings.

FIG. 12A provides a photograph of Pluronic® F127/W379-PCL core-shell nanofiber membranes. FIGS. 12B-12D provide SEM images of Pluronic® F127-PCL core-shell nanofibers, Pluronic® F127/W379-PCL core-shell nanofibers, and Pluronic® F127/W379-PCL-S nanofibers, respectively.

FIG. 13 provide a graph of the in vitro release profiles of the W379 peptide from F127/W379-PCL core-shell nanofibers and after electrospray deposition of W379 (F127/W379-PCL-S).

FIG. 14 provides a graph of an in vitro antibacterial efficacy test of F127/W379-PCL core-shell nanofibers. The bacterial solution was diluted into 1.0×10⁷ CFU/mL in PBS. One milligram of PCL or F127/W379-PCL core-shell nanofiber membranes was co-incubated with the bacterial solution for 2 hours at 37° C. Total remaining bacteria were determined by culturing on agar plates.

FIG. 15 shows the in vitro cytotoxicity test of W379 peptide-loaded PCL nanofiber membranes. AlamarBlue® cell viability tests against HaCaT cells and U937 cells are shown. Each data point represents arithmetic mean±SD values from four samples.

FIG. 16 provides results from in vivo antibiofilm efficacy tests of F127/W379-PCL-S nanofiber dressings. The MRSA and biofilm-containing chronic wounds created in type II diabetic mice were treated by F127/W379-PCL-S nanofiber dressings without and with debridement. PCL: Pluronic® F127-PCL core-shell nanofiber membranes. W379 peptide/PCL: W379/Pluronic® F127-PCL core-shell nanofiber membranes. Debridement PCL: the wounds were debrided and treated with Pluronic® F127-PCL core-shell nanofiber membranes. Debridement W379 peptide/PCL: the wounds were debrided and treated with W379/Pluronic® F127-PCL core-shell nanofiber membranes. Without treatment: no treatment was applied to the wounds.

FIG. 17A provides a photo of a Janus-type dressing. Inset (left bottom): SEM image showing the whole view of a microneedle array immobilized on the surface of nanofiber membrane. Scale bar=1 mm. Inset (right top): SEM image showing the electrospun nanofiber substrate. Scale bar=5 μm. FIG. 17B: SEM image showing peptides containing dissolvable microneedle arrays on nanofiber membranes (100 microneedles per membrane). FIG. 17C: SEM image showing magnified image of FIG. 17B and one microneedle was intentionally removed to reveal the nanofiber matrices underneath. FIG. 17D: SEM image showing peptides containing dissolvable microneedle arrays on nanofiber membranes (150 microneedles per membrane).

FIG. 18 provides a graph of in vitro antibacterial activity. F127-PCL: Pluronic® F127-PCL core-shell nanofiber membranes. W379/F127-PCL: W379/Pluronic® F127-PCL core-shell nanofiber membranes. W379/F127-PCL+W379/PVP MN: Janus-type antimicrobial dressing consisting of W379/Pluronic® F127-PCL core-shell nanofiber membranes and W379 loaded PVP microneedle arrays.

FIGS. 19A-19C provide SEM images show the morphology of A. baumanni, P. aeruginosa, and MRSA biofilms on the excisional wounds in human skin explants. Inset of FIG. 19A: excisional wounds covered by Janus-type antimicrobial dressings (6 mm in diameter) in human skin explants. FIG. 19D: Quantification of bacteria biofilms on excisional wounds in human skin explants.

FIGS. 20A-20D show the biofilm treatment efficacy of Janus-type antimicrobial dressings in an ex vivo biofilm-containing human skin wound model. FIG. 20A: The

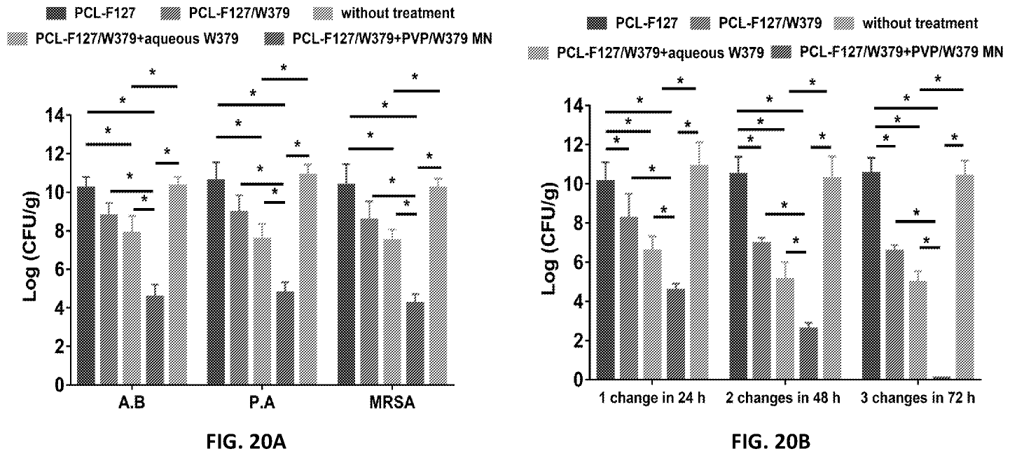

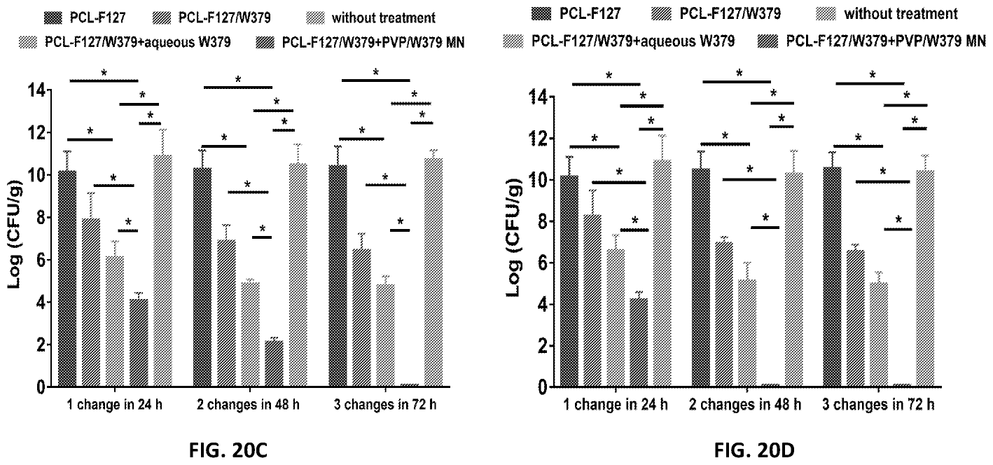

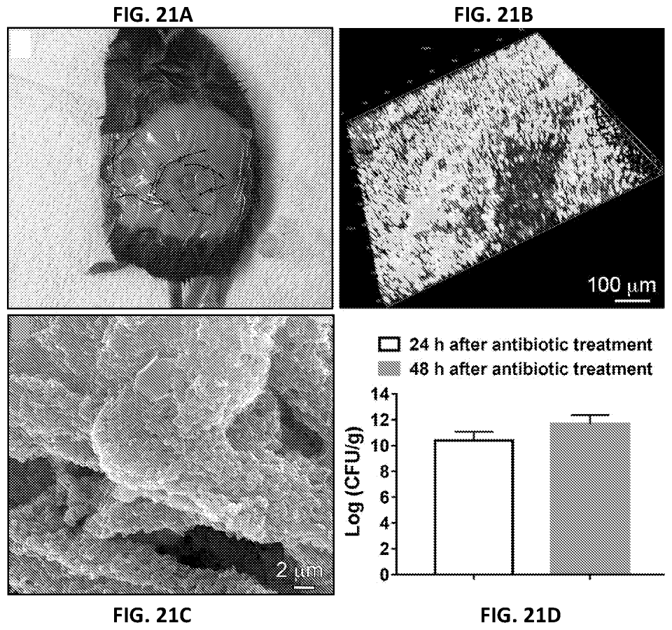

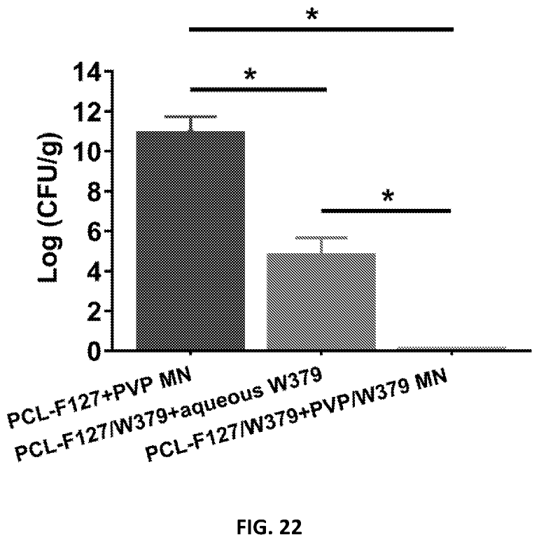

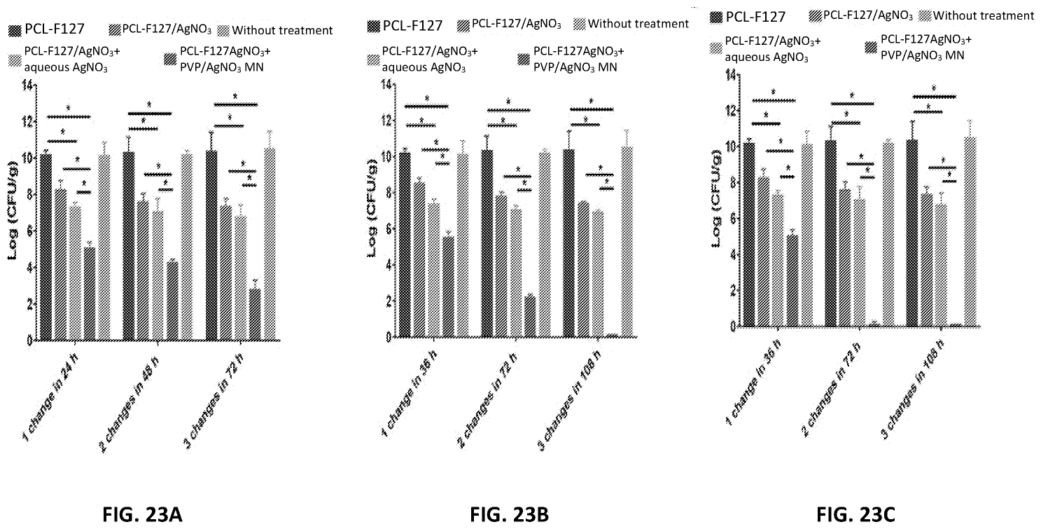

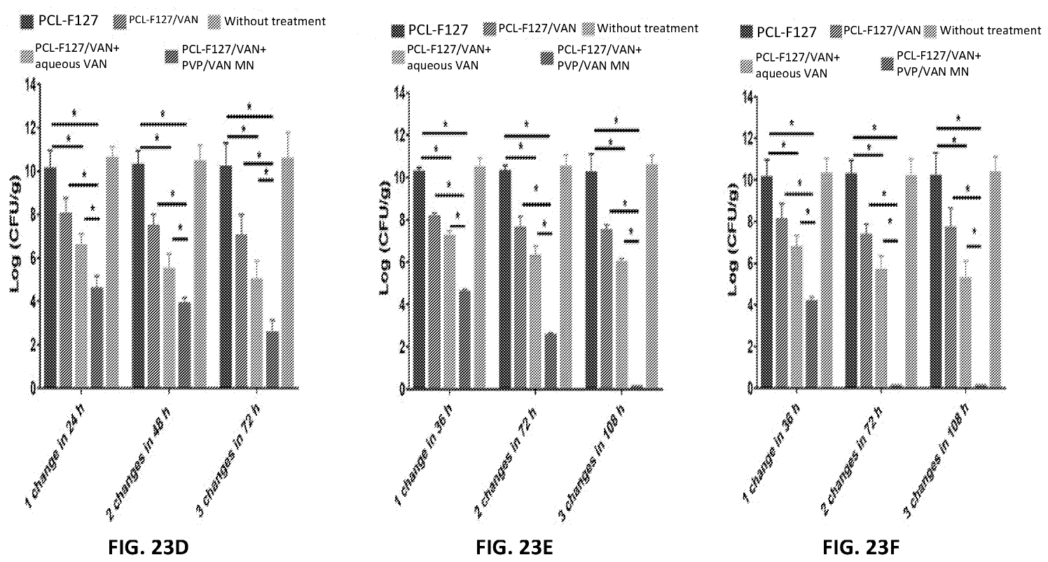

Janus-type dressing was not changed within 24 h against three different pathogens. FIG. 20B: Different dressing changes against MRSA biofilm. FIG. 20C: Double the peptide concentrations in microneedles against MRSA biofilm. FIG. 20D: High density microneedles in Janus-type dressings against MRSA biofilm. PCL-F127: PCL-F127 nanofibers. PCL-F127/W379: W379 peptides loaded PCL-F127 nanofibers. PCL-F127/W379+aqueous W379: W379 peptides loaded PCL-F127 nanofibers+free W379 peptides. PCL-F127/W379+PVP/W379 MN: Janus-type dressing composed of W379 peptides loaded PCL-F127 nanofiber membrane and W379 peptides-loaded microneedle arrays. Without treatment: no treatment to the wounds.

FIG. 21A provides an image of a type II diabetic mice wound. Wounds were created and fixed with a splint, and MRSA was inoculated for 24 hours. FIG. 21B: LIVE/DEAD® staining for the tissue collected from wounds after 24 hours of MRSA inoculation and subsequent 24 hours 2% mupirocin treatment. FIG. 21C: SEM observation of the tissue collected from wounds after 24 hours of MRSA inoculation and subsequent 24 hours 2% mupirocin treatment. FIG. 21D: Quantification of bacterial load in the wound after 24 hours and 48 hours of MRSA inoculation and subsequent 24 hours of 2% mupirocin treatment.

FIG. 22 provides a graph of the anti-biofilm efficacy of Janus-type antimicrobial dressings in vivo. The dressings were changed every 24 hours for 3 times.

FIGS. 23A-23F show the ex vivo efficacy of silver and vancomycin loaded Janus-type dressings. FIG. 23A: AgNO₃-loaded Janus-type dressings with low density of microneedles were changed every 24 hours for one to three times. FIG. 23B: AgNO₃-loaded Janus-type dressings with low density of microneedles were changed every 36 hours for one to three times. FIG. 23C: AgNO₃-loaded Janus-type dressings with high density of microneedles were changed every 36 hours for one to three times. FIG. 23D: Vancomycin-loaded Janus-type dressings with low density of microneedles were changed every 24 hours for one to three times. FIG. 23E: Vancomycin-loaded Janus-type dressings with low density of microneedles were changed every 36 hours for one to three times. FIG. 23F: Vancomycin-loaded Janus-type dressings with high density of microneedles were changed every 36 hours for one to three times. PCL-F127: PCL-F127 nanofibers. PCL-F127/AgNO₃: AgNO₃-loaded PCL-F127 nanofibers. PCL-F127/AgNO₃+aqueous AgNO₃: AgNO₃-loaded PCL-F127 nanofibers+free AgNO₃ peptides. PCL-F127/AgNO₃+PVP/AgNO₃ MN: Janus-type dressing composed of AgNO₃-loaded PCL-F127 nanofiber membrane and AgNO₃-loaded microneedle arrays. Without treatment: no treatment to the wounds. PCL-F127/VAN: Vancomycin-loaded PCL-F127 nanofibers. PCL-F127/VAN+aqueous VAN: Vancomycin-loaded PCL-F127 nanofibers+free vancomycin. PCL-F127/VAN+PVP/VAN MN: Janus-type dressing composed of vancomycin-loaded PCL-F127 nanofiber membrane and vancomycin-loaded microneedle arrays.

### DETAILED DESCRIPTION OF THE INVENTION

Peptides have been widely used in biomedical applications. The antimicrobial peptide LL-37, the only cathelicidin-derived host defense peptide found in humans, effectively kills multi-drug resistant pathogens (Steinstraesser, et al. (2011) Immunobiology, 216(3):322-333). LL-37 is a key component of the innate immune system and represents the first line of defense against various invading pathogens (Chieosilapatham, et al. (2018) Cur. Pharm. Design, 24(10):1079-1091). Although direct application of LL-37 or over-expression by vectors increases local concentrations, significant problems with delivery, host-cell toxicity, tissue destruction and inflammation exist (Li, et al. (2013) PloS One, 8(2):e56616). Furthermore, the antimicrobial efficacy of LL-37 is heavily dependent on the environment including pH, salt/ions concentration, and proteases (Xhindoli, et al. (2016) Biochim. Biophys. Acta-Biomembranes, 1858(3):546-566). In fact, LL-37 peptides present low antimicrobial activities under serum and tissue conditions (Hancock, et al. (2016) Nat. Rev. Immunol., 16(5):321-344). LL-37 inhibits biofilm growth of Staphylococcus aureus USA300 in vitro only at a low concentration (Haisma, et al. (2014) Antimicrob. Agents Chemother., 58(8):4411-4419). LL-37 was unable to inhibit bacterial attachment or disrupt preformed biofilms (Mishra, et al. (2016) ACS Med. Chem. Lett., 7:117-121). Towards this end, human cathelicidin LL-37 has been successfully engineered into selective, stable and potent antimicrobial peptides, which display superior antibiofilm capability (Mishra, et al. (2016) ACS Med. Chem. Lett., 7:117-121; Wang, et al. (2014) Biochim. Biophys. Acta-Biomembranes, 1838(9):2160-2172).

Electrospinning is a versatile technique for generating long fibers with nanoscale diameters (Romano, et al. (2016) Mol. Pharm., 13(3):729-736). Electrospun nanofiber wound dressings offer significant advantages over hydrogels or sponges for local drug delivery (Chen, et al. (2016) Mol. Pharm., 13(12):4152-4167). Collagen, fibrin, poly(ethylene glycol)(PEG), and alginate hydrogels are capable of soft tissue-like compliance, but are difficult to suture and are often too weak to support physiologic loads (Wang, et al. (2011) Mol. Pharm., 8(4):1025-1034; Spicer, et al. (2010) J. Control. Release, 148(1):49-55; Knop, et al. (2010) Angew. Chem. Int. Edit., 49(36):6288-6308; Augst, et al. (2006) Macromol. Biosci., 6(8):623-633). In addition, it is difficult to encapsulate hydrophobic molecules inside hydrogels (Hoare, et al. (2008) Polymer, 49(8):1993-2007). In sponges, hydrophobic drug molecules are usually crystalized after encapsulation, which slows down the dissolution rate and is unfavorable. On the other hand, electrospun nanofibers serve as ideal materials for topical drug delivery with at least the following unique characteristics: i) ease of incorporation of drugs, including hydrophobic molecules inside nanofibers, ii) ease of control of release profiles by controlling the porosity of nanofibers and the degradation profiles, iii) exhibiting an amorphous state for encapsulated hydrophobic drug molecules and thus enhanced solubility of drugs (Khansari, et al. (2013) Mol. Pharm., 10(12):4509-4526). The architecture of electrospun nanofibers mimics the collagen structure of the extracellular matrix (ECM)-a 3D network of collagen fibers 50-500 nm in diameter. Therefore, compared to traditional wound dressings, nanofiber-based wound dressings provide several functional and structural advantages including hemostasis, high filtration, semi-permeability, conformability and scar-free healing (Sundaran, et al. (2017) J. Mater. Sci. Mater. Med., 28(6):88). Even though electrospun nanofibers offer numerous advantages, their full potential has not been realized. Their antimicrobial use is limited to the surface modifications with chitosan, Ag and ZnO nanoparticles, and encapsulation of antibiotics and Ag nanoparticles/ions (Wang, et al. (2012) Carbohyd. Polym., 90(1):8-15; Shafei, et al. (2011) Carbohyd. Polym., 83(2):920-925; Li, et al. (2005) Nanotechnology, 16(9):1912). Herein, the potential of these materials for fighting infections is increased by developing nanofiber wound dressings for local delivery of antibiotics, particularly antimicrobial peptides such as LL-37 engineered antimicrobial peptides. Topical delivery of molecularly engineered antimicrobial peptides from nanofiber wound dressings will effectively treat multidrug resistant bacteria caused infections in wounds, including chronic wounds.

Biofilms of multidrug resistant bacteria post a great challenge in wound care. In particular, biofilms in chronic wounds, including diabetic foot ulcers, pressure ulcers, and venous leg ulcers, pose a major challenge to wound management. Herein, topical delivery is shown of molecularly engineered antimicrobial peptides using electrospun nanofiber dressings as carrier for treatment of biofilms of multidrug resistant bacteria in diabetic wounds. The molecularly engineered human cathelicidin peptides 17BIPHE2 was successfully encapsulated in the core of Pluronic® F127/17BIPHE2-PCL core-shell nanofibers. The in vitro release profiles of 17BIPHE2 showed an initial burst followed by a sustained release over 4 weeks. The peptide nanofiber formulations effectively killed MRSA USA300. Similarly, the 17BIPHE2 peptide containing nanofibers could effectively kill other bacteria including Klebsiella pneumonia (10⁴-10⁶ CFU inoculation) and Acinetobacter baumannii (10⁴-10⁷ CFU inoculation) clinical strains in vitro without showing evident cytotoxicity to skin cells and monocytes. Importantly, 17BIPHE2-containing nanofiber dressings caused a five-magnitude decrease of the MRSA USA300 CFU in a biofilm-containing chronic wound model based on type II diabetic mice. In combination with debridement, 17BIPHE2-containing nanofiber dressings could completely eliminate the biofilms, providing one possible solution to chronic wound treatment. Taken together, the biodegradable nanofiber-based wound dressings developed in this study can be utilized to effectively deliver antimicrobials (e.g., antibiotics) such as molecularly engineered peptides to treat biofilm-containing chronic wounds.

In addition to the above, a novel Janus-type antimicrobial dressing is provided comprising antimicrobial (e.g., antibiotic)-loaded, particularly molecularly engineered peptides-loaded, electrospun nanofiber membranes and dissolvable microneedle arrays for the treatment of biofilms in chronic wounds. Molecularly engineered peptides-loaded nanofibers exhibited high efficacy against a broad spectrum of pathogens including antibiotic resistant bacteria in vitro. The Janus-type dressings can eradicate biofilms in both an ex vivo human skin wound infection models and a type II diabetic mice wound infection model by simply changing the dressings without surgical debridement. The results indicate the Janus-type antimicrobial dressings is an effective intervention for the management of biofilms in chronic wounds.

In accordance with the instant invention, antimicrobial (e.g., antibiotic)-loaded nanofiber structures/compositions are provided. In a particular embodiment, the antimicrobial (e.g., antibiotic)-loaded nanofiber structure is a mat or two-dimensional structure. In a particular embodiment, the antimicrobial (e.g., antibiotic)-loaded nanofiber structure is in the form of a wound dressing. The antimicrobial (e.g., antibiotic)-loaded nanofiber structure may be in any form including, without limitation, a wound dressing, bandage, gauze, covering, suture, thread, ligature, hemostasis material, or coating for biomedical device or implant. In a particular embodiment, the antimicrobial (e.g., antibiotic)-loaded nanofiber structure is in a wound dressing.

The nanofibers of the instant invention can be fabricated by any method. In a particular embodiment, the nanofiber structures comprise electrospun nanofibers. The nanofiber structure may comprise aligned fibers (e.g., uniaxially aligned), random fibers, and/or entangled fibers. In a particular embodiment, the nanofiber structure comprises random fibers. In a particular embodiment, the nanofiber structure comprises aligned fibers (e.g., uniaxially, radially, vertically, or horizontally). In a particular embodiment, the nanofiber structure is a porous nanofiber sponge or aerogel (e.g., Weng et al. (2018) Adv. Healthcare Mater., 7(10): 1701415). While the application generally describes nanofibers (fibers having a diameter less than about 1 μm (e.g., average diameter)) structures, the instant invention also encompasses microfibers (fibers having a diameter greater than about 1 μm (e.g., average diameter)) structures. The nanofiber structures may be of any shape and can be synthesized in the desired shape (e.g., with a mold) or modified after synthesis (e.g., by cutting or other manipulation).

Generally, the synthesized nanofiber structures (e.g., by electrospinning) is used as synthesized (e.g., as a nanofiber mat or other two-dimensional structure). However, the instant invention also encompasses expanded nanofiber structures. Methods for expanding nanofiber mats are described, for example, in U.S. Patent Application Publication No. 20170296703 and WO 2019/060393, each incorporated by reference herein. In certain embodiments, the nanofiber mat is expanded into an expanded nanofiber structure by exposing the nanofiber mat to gas bubbles. The bubbles can be generated by chemical reactions or physical manipulations. For example, the nanofiber mat can be submerged or immersed in a bubble/gas producing chemical reaction or physical manipulation. Generally, the longer the exposure to the bubbles, the greater the thickness and porosity of the expanded nanofiber structure increases. The nanofiber mat may also be expanded within a mold (e.g., a metal, plastic, or other material that does not expand in the presence of gas bubbles) to assist in the formation of a desired shape. The nanofiber mat may be treated with air plasma prior to exposure to gas bubbles (e.g., to increase hydrophilicity).

After exposure to the bubbles, the expanded nanofiber structure may be washed and/or rinsed in water and/or a desired carrier or buffer (e.g., a pharmaceutically or biologically acceptable carrier). Trapped gas bubbles may be removed by applying a vacuum to the expanded nanofiber structure. For example, the expanded nanofiber structure may be submerged or immersed in a liquid (e.g., water and/or a desired carrier or buffer) and a vacuum may be applied to rapidly remove the gas bubbles. After expansion (e.g., after rinsing and removal of trapped gas), the expanded nanofiber structure may be placed in storage in cold solution or lyophilized and/or freeze-dried.

The gas bubbles of the instant invention can be made by any method known in the art. The bubbles may be generated, for example, by chemical reactions or by physical approaches. Electrospun nanofiber mats can be expanded in the third dimension with ordered structures using gas bubbles generated by chemical reactions in an aqueous solution (see, e.g., WO 2016/053988; WO 2019/060393; Jiang et al. (2018) Acta Biomater., 68:237-248; Jiang, et al. (2015) ACS Biomater. Sci. Eng., 1:991-1001; Jiang, et al. (2016) Adv. Healthcare Mater., 5:2993-3003; Joshi, et al. (2015) Chem. Eng. J., 275:79-88; each of the foregoing incorporated by reference herein). In a particular embodiment, the chemical reaction or physical manipulation does not damage or alter or does not substantially damage or alter the nanofibers (e.g., the nanofibers are inert within the chemical reaction and not chemically modified). As explained hereinabove, the nanofiber mat may be submerged or immersed in a liquid comprising the reagents of the bubble-generating chemical reaction. Examples of chemical reactions that generate bubbles include, without limitation:

NaBH₄+2H₂O═NaBO₂+4H₂

NaBH₄+4H₂O═4H₂(g)+H₃BO₃+NaOH

HCO₃⁻+H⁺═CO₂+H₂O

NH₄⁺+NO₂⁻═N₂+2H₂O

H₂CO₃═H₂O+CO₂

2H⁺+S²⁻═H₂S

2H₂O₂═O₂+2H₂O

3HNO₂=2NO+HNO₃+H₂O

HO₂CCH₂COCH₂CO₂H═2CO₂+CH₃COCH₃

2H₂O₂=2H₂+O₂

CaC₂+H₂O═C₂H₂

Zn+2HCl═H₂ZnCl₂

2KMnO₄+16HCl=2KCl+2MnCl₂+H₂O+5Cl₂

In a particular embodiment, the chemical reaction is the hydrolysis of NaBH₄ (e.g., NaBH₄+2H₂O═NaBO₂+4H₂). In a particular embodiment, CO₂ gas bubbles (generated chemically or physically) are used (e.g., for hydrophilic polymers).

Examples of physical approaches for generating bubbles of the instant invention include, without limitation: 1) create high pressure (fill gas)/heat in a sealed chamber and suddenly reduce pressure; 2) dissolve gas in liquid/water in high pressure and reduce pressure to release gas bubbles; 3) use supercritical fluids (reduce pressure) like supercritical CO₂; 4) use subcritical gas liquid (then reduce pressure) (e.g., liquid CO₂, liquid propane and isobutane); 5) fluid flow; 6) apply acoustic energy or ultrasound to liquid/water; 7) apply a laser (e.g., to a liquid or water); 8) boiling; 9) reduce pressure boiling (e.g., with ethanol); and 10) apply radiation (e.g., ionizing radiation on liquid or water). The nanofiber mat may be submerged or immersed in a liquid of the bubble-generating physical manipulation.

In a particular embodiment, the nanofiber mats are expanded using a subcritical or supercritical fluid or liquid (e.g., CO₂, N₂, N₂O, hydrocarbons, and fluorocarbons). In a particular embodiment, liquid CO₂ is utilized. For example, nanofiber mats may be expanded by exposing to, contacting with or being placed into (e.g., submerged or immersed) a subcritical liquid/fluid (e.g., subcritical CO₂) and then depressurized. The cycle of placing the nanofibrous structures into subcritical CO₂ and depressurizing may be performed one or more times. Generally, the more times the expansion method is used the thickness and porosity of the nanofibrous (or microfibrous) structure increases. For examples, the cycle of exposure to subcritical CO₂ and then depressurization may be performed one, two, three, four, five, six, seven, eight, nine, ten, or more times, particularly 1-10 times, 1-5 times, or 1-3 times. In a particular embodiment, the cycle of exposure to subcritical CO₂ and then depressurization is performed at least 2 times (e.g., 2-10 times, 2-5 times, 2-4 times, or 2-3 times). In a particular embodiment, the method comprises placing the nanofibrous mat and dry ice (solid CO₂) in a sealed container, allowing the dry ice to turn into liquid CO₂, and then unsealing the container to allow depressurization.

The nanofiber mat and subcritical fluid (e.g., subcritical CO₂; or solid form of subcritical fluid (e.g., dry ice)) may be contained in any suitable container (e.g., one which can withstand high pressures). For example, the subcritical fluids and the nanofiber mat may be contained within, but not limited to: chambers, vessels, reactors, chambers, and tubes. In a particular embodiment, the equipment or container used during the methods of the present invention will have a feature or component that allows control of the depressurization rate of the subcritical fluid. Depressurization of the subcritical fluid can be done using a variety of methods including but not limited to manually opening the container to decrease pressure or by using some type of equipment that can regulate the rate of depressurization of the reaction vessel.

In a particular embodiment, the nanofibers of the instant invention have a core-shell morphology. Generally, at least the core of the nanofiber comprises the antimicrobial/antibiotic (e.g., antimicrobial peptide). In a particular embodiment, the core is hydrophilic and the shell is hydrophobic. In a particular embodiment, the core is hydrophobic and the shell is hydrophilic. In a particular embodiment, the core is hydrophilic and comprises the hydrophilic portion of an amphiphilic polymer (e.g., amphiphilic block copolymer), the antimicrobial/antibiotic (e.g., antimicrobial peptide), and optionally a hydrophilic polymer and the shell is hydrophobic and comprises the hydrophobic portion of the amphiphilic polymer (e.g., amphiphilic block copolymer) and a hydrophobic polymer. The core-shell nanofibers may be synthesized, for example, using co-axial electrospinning. In a particular embodiment, the synthesis comprises performing co-axial electrospinning using two inputs wherein one input comprises an aqueous phase/solution and the other input comprises a polymer phase/solution. The aqueous phase may comprise, for example, a water-soluble antimicrobial/antibiotic (e.g., antimicrobial peptide) and an amphiphilic polymer (e.g., an amphiphilic block copolymer such as a poloxamer (e.g., poloxamer 407)). The polymer phase may comprise, for example, a hydrophobic polymer (e.g., PCL).

The nanofibers of the instant invention may comprise any polymer. In a particular embodiment, the polymer is biocompatible. The polymer may be biodegradable or non-biodegradable. In a particular embodiment, the polymer is a biodegradable polymer. In a particular embodiment, the polymer is FDA approved. The polymers of the nanofibers may by hydrophobic, hydrophilic, or amphiphilic. In a particular embodiment, the nanofiber comprises a hydrophobic polymer. In a particular embodiment, the nanofiber comprises a hydrophilic polymer. The polymers of the nanofiber may be, for example, a homopolymer, random copolymer, blended polymer, copolymer, or a block copolymer. Block copolymers are most simply defined as conjugates of at least two different polymer segments or blocks. The polymer may be, for example, linear, star-like, graft, branched, dendrimer based, or hyper-branched (e.g., at least two points of branching). The polymer of the invention may have from about 2 to about 10,000, about 2 to about 1000, about 2 to about 500, about 2 to about 250, or about 2 to about 100 repeating units or monomers. The polymers of the instant invention may comprise capping termini.

Examples of hydrophobic polymers include, without limitation: polyvinyl alcohol (PVA), poly(hydroxyethyl methacrylate), poly(N-isopropyl acrylamide), poly(lactic acid) (PLA (or PDLA)), poly(lactide-co-glycolide) (PLG), poly(lactic-co-glycolic acid) (PLGA), polyglycolide or polyglycolic acid (PGA), polycaprolactone (PCL), poly(aspartic acid), polyoxazolines (e.g., butyl, propyl, pentyl, nonyl, or phenyl poly(2-oxazolines)), polyoxypropylene, poly(glutamic acid), poly(propylene fumarate) (PPF), poly(trimethylene carbonate), polycyanoacrylate, polyurethane, polyorthoesters (POE), polyanhydride, polyester, poly(propylene oxide), poly(caprolactonefumarate), poly(l,2-butylene oxide), poly(n-butylene oxide), poly(ethyleneimine), poly(tetrahydrofurane), ethyl cellulose, polydipyrolle/dicabazole, starch, polyvinylidene fluoride (PVDF), polytetrafluoroethylene (PTFE), polydioxanone (PDO), polyether poly(urethane urea) (PEUU), cellulose acetate, polypropylene (PP), polyethylene terephthalate (PET), nylon (e.g., nylon 6), polycaprolactam, PLA/PCL, poly(3-hydroxybutyrate-co-3-hydroxyvalerate) (PHBV), PCL/calcium carbonate, and/or poly(styrene).

Examples of hydrophilic polymers include, without limitation: polyvinylpyrrolidone (PVP), poly(ethylene glycol) and poly(ethylene oxide) (PEO), chitosan, collagen, chondroitin sulfate, sodium alginate, gelatin, elastin, hyaluronic acid, silk fibroin, sodium alginate/PEO, silk/PEO, silk fibroin/chitosan, hyaluronic acid/gelatin, collagen/chitosan, chondroitin sulfate/collagen, and chitosan/PEO.

Amphiphilic copolymers or polymer composites may comprise a hydrophilic polymer (e.g., segment/block) and a hydrophobic polymer (e.g., segment/block) from those listed above (e.g., gelatin/polyvinyl alcohol (PVA), PCL/collagen, chitosan/PVA, gelatin/elastin/PLGA, PDO/elastin, PHBV/collagen, PLA/hyaluronic acid, PLGA/hyaluronic acid, PCL/hyaluronic acid, PCL/collagen/hyaluronic acid, gelatin/siloxane, PLLA/MWNTs/hyaluronic acid).

Examples of polymers particularly useful for electrospinning are provided in Xie et al. (Macromol. Rapid Commun. (2008) 29:1775-1792; incorporated by reference herein; see e.g., Table 1). Examples of compounds or polymers for use in the fibers of the instant invention, particularly for electrospun nanofibers include, without limitation: natural polymers (e.g., chitosan, gelatin, collagen type I, II, and/or III, elastin, hyaluronic acid, cellulose, silk fibroin, phospholipids (Lecithin), fibrinogen, hemoglobin, fibrous calf thymus Na-DNA, virus M13 viruses), synthetic polymers (e.g., PLGA, PLA, PCL, PHBV, PDO, PGA, PLCL, PLLA-DLA, PEUU, cellulose acetate, PEG-b-PLA, EVOH, PVA, PEO, PVP), blended (e.g., PLA/PCL, gelatin/PVA, PCL/gelatin, PCL/collagen, sodium aliginate/PEO, chitosan/PEO, Chitosan/PVA, gelatin/elastin/PLGA, silk/PEO, silk fibroin/chitosan, PDO/elastin, PHBV/collagen, hyaluronic acid/gelatin, collagen/chondroitin sulfate, collagen/chitosan), and composites (e.g., PDLA/HA, PCL/CaCO₃, PCL/HA, PLLA/HA, gelatin/HA, PCL/collagen/HA, collagen/HA, gelatin/siloxane, PLLA/MWNTs/HA, PLGA/HA). In a particular embodiment, the nanofiber comprises polymethacrylate, poly vinyl phenol, polyvinylchloride, cellulose, polyvinyl alcohol, polyacrylamide, PLGA, collagen, polycaprolactone, polyurethanes, polyvinyl fluoride, polyamide, silk, nylon, polybennzimidazole, polycarbonate, polyacrylonitrile, polyvinyl alcohol, polylactic acid, polyethylene-co-vinyl acetate, polyethylene oxide, polyaniline, polystyrene, polyvinylcarbazole, polyethylene terephthalate, polyacrylic acid-polypyrene methanol, poly(2-hydroxyethyl methacrylate), polyether imide, polyethylene glycol, poly(ethylene-co-vinyl alcohol), polyacrylnitrile, polyvinyl pyrrolidone, polymetha-phenylene isophthalamide, gelatin, chitosan, starch, pectin, cellulose, methylcellulose, sodium polyacrylate, starch-acrylonitrile co-polymers, and/or combinations of two or more polymers. In a particular embodiment, the nanofiber comprises polycaprolactone (PCL).

In a particular embodiment, the nanofiber an amphiphilic block copolymer, particularly an amphiphilic block copolymer comprising hydrophilic poly(ethylene oxide) (PEO) and hydrophobic poly(propylene oxide) (PPO). In a particular embodiment, the nanofiber comprises a poloxamer or an amphiphilic triblock copolymer comprising a central hydrophobic PPO block flanked by two hydrophilic PEO blocks (i.e., an A-B-A triblock structure). In a particular embodiment, the amphiphilic block copolymer is selected from the group consisting of Pluronic® L31, L35, F38, L42, L44, L61, L62, L63, L64, P65, F68, L72, P75, F77, L81, P84, P85, F87, F88, L92, F98, L101, P103, P104, P105, F108, L121, L122, L123, F127, 10R5, 10R8, 12R3, 17R1, 17R4, 17R8, 22R4, 25R1, 25R2, 25R4, 25R5, 25R8, 31R1, 31R2, and 31R4. In a particular embodiment, the nanofiber comprises poloxamer 407 (Pluronic® F127). The amphiphilic block copolymer (e.g., poloxamer) may be added in various amounts to the polymer solution during the synthesis process (e.g., electrospinning). In a particular embodiment, 0% to 20%, particularly 0% to 10%, of the polymer solution is amphiphilic block copolymer (e.g., poloxamer). In a particular embodiment, 0.1% to 5%, particularly 0.5% to 2%, of the polymer solution is amphiphilic block copolymer (e.g., poloxamer). In a particular embodiment, the polymer solution contains 10% polymer (e.g., PCL).

In a particular embodiment, the nanofibers and/or nanofiber structures are further coated with the antimicrobial/antibiotic (e.g., antimicrobial peptide). In a particular embodiment, the nanofibers and/or nanofiber structures are coated with the contents of the core of the nanofibers. In a particular embodiment, the nanofibers and/or nanofiber structures are coated with the antimicrobial/antibiotic (e.g., antimicrobial peptide) and the amphiphilic polymer (e.g., amphiphilic block copolymer (e.g., poloxamer)). The antimicrobial/antibiotic coating can be applied to the nanofibers and/or nanofiber structures by, for example, electrospraying (e.g., with an aqueous solution comprising the antimicrobial/antibiotic and the amphiphilic polymer (e.g., amphiphilic block copolymer (e.g., poloxamer)).

In a particular embodiment, the nanofibers and/or nanofiber structures are coated with additional materials to enhance their properties. For example, the nanofibers and/or nanofiber structure may be coated with proteins, collagen, fibronectin, collagen, a proteoglycans, elastin, or a glycosaminoglycans (e.g., hyaluronic acid, heparin, chondroitin sulfate, or keratan sulfate). In a particular embodiment, the nanofiber structures comprise a material that enhances the nanofiber structure's ability to absorb fluids, particularly aqueous solutions (e.g., blood). In a particular embodiment, the nanofibers comprise a material which enhances the absorption properties. In a particular embodiment, the nanofibers and/or nanofiber structures are coated with the material which enhances the absorption properties. Materials which enhance the absorption properties of the nanofiber structures include, without limitation: gelatin, alginate, chitosan, collagen, starch, pectin, cellulose, methylcellulose, sodium polyacrylate, starch-acrylonitrile co-polymers, other natural or synthetic hydrogels, and derivatives thereof (e.g., del Valle et al., Gels (2017) 3:27). In a particular embodiment, the material is a hydrogel (e.g., a polymer matrix able to retain water, particularly large amounts of water, in a swollen state). In a particular embodiment, the material is gelatin. In a particular embodiment, the nanofiber structures are coated with about 0.05% to about 10% coating material (e.g., gelatin), particularly about 0.1% to about 10% coating material (e.g., gelatin) or about 0.1% to about 1% coating material (e.g., gelatin). In a particular embodiment, the material (e.g., hydrogel) is crosslinked.

The term “coat” refers to a layer of a substance/material on the surface of a structure. Coatings may, but need not, also impregnate the nanofiber structure. Further, while a coating may cover 100% of the nanofibers and/or nanofiber structure, a coating may also cover less than 100% of the surface of the nanofibers and/or nanofiber structure (e.g., at least about 75%, at least about 80%, at least about 85%, at least about 90%, at least about 95%, at least about 98%, or more the surface may be coated).

In a particular embodiment, the nanofibers and/or nanofiber structures are mineralized (e.g., comprise minerals and/or coated with minerals). Mineralization, for example, with hydroxyapatite, can enhance the adhesion of osteogenic precursor cells in vitro and in vivo (Duan, et al., Biomacromolecules (2017) 18:2080-2089). In a particular embodiment, the nanofibers and/or nanofiber structures are coated with Ca, P, and/or O. In a particular embodiment, the nanofibers and/or nanofiber structures are coated with hydroxyapatite, fluorapatite, and/or chlorapatite, particularly hydroxyapatite. In a particular embodiment, the nanofibers and/or nanofiber structures are immersed in simulated body fluid (SBF) for mineralization (e.g., a solution comprising NaCl, CaCl₂, NaH₂PO₄, and NaHCO₃).

In a particular embodiment, the nanofiber structures of the instant invention are crosslinked (e.g., before or after expansion). Crosslinking may be done using a variety of techniques including thermal crosslinking, chemical crosslinking, and photo-crosslinking. For example, the nanofiber structures of the instant invention may be crosslinked with a crosslinker such as, without limitation: formaldehyde, paraformaldehyde, acetaldehyde, glutaraldehyde, a photocrosslinker, genipin, and natural phenolic compounds (Mazaki, et al., Sci. Rep. (2014) 4:4457; Bigi, et al., Biomaterials (2002) 23:4827-4832; Zhang, et al., Biomacromolecules (2010) 11:1125-1132; incorporated herein by reference). The crosslinker may be a bifunctional, trifunctional, or multifunctional crosslinking reagent. In a particular embodiment, the crosslinker is glutaraldehyde.

The nanofiber structures of the instant invention may also be sterilized. For example, the nanofiber structures can be sterilized using various methods (e.g., by treating with ethylene oxide (e.g., gas), gamma irradiation, or 70% ethanol).

As stated hereinabove, the nanofiber and/or nanofiber structures of the instant invention comprise or encapsulate at least one antimicrobial (e.g., antibacterial, antibiotic, antiviral, and/or antifungal), particularly an antibiotic. In a particular embodiment, the antimicrobial is hydrophilic. The antimicrobial may be added to the nanofiber structures during synthesis and/or after synthesis. The antimicrobial may be conjugated to the nanofiber structure and/or coating material, encapsulated by the nanofiber structure, and/or coated on the nanofiber structure (e.g., with, underneath, and/or on top of the coating that enhances the nanofiber structure's ability to absorb fluids). In a particular embodiment, the antimicrobial is not directly conjugated to the nanofiber structure (e.g., encapsulated).

Antimicrobials may include, without limitation, small molecules, peptides, proteins, DNA, RNA, and other known biologic substances. In a particular embodiment, the antimicrobial is a small molecule. In a particular embodiment, the antimicrobial is an antiviral, antifungal, antibiotic or antibacterial, particularly an antibiotic or antibacterial.

Examples of antimicrobials include, without limitation, antibiotics such as beta-lactams (e.g., penicillin, ampicillin, oxacillin, cloxacillin, methicillin, cephalosporin, etc.), monobactams (e.g., aztreonam, tigemonam, nocardicin A, tabtoxin, etc.), carbapenems (e.g., imipenem, meropenem, ertapenem, doripenem, etc.), cephalosporins (e.g., cefdinir, cefaclor, cephalexin, cefixime, cefepime, etc.), carbacephems, cephamycins, macrolides (e.g., erythromycin, clarithromycin, azithromycin etc.), quinolones or fluoroquinolones (e.g., ciprofloxacin, levofloxacin, ofloxacin, delafloxacin, etc.), tetracyclines (e.g., tetracycline, doxycycline etc.), sulfonamides (e.g., sulfamethoxazole, sulfafuraxole, etc.), aminoglycosides (e.g., gentamicin, neomycin, tobramycin, kanamycin, etc.), oxazolidinones (e.g., linezolid, posizolid, tedizolid, radezolid, contezolid, etc.), lipopeptides (e.g., daptomycin), glycylcyclines (e.g., tigecycline), moenomycins, aminocoumarins (e.g., novobiocin), co-trimoxazoles (e.g., trimethoprim and sulfamethoxazole), lincosamides (e.g., clindamycin and lincomycin), polypeptides (e.g., colistin), and glycopeptides (e.g., vancomycin); silver containing compounds (e.g., silver ions, silver nitrate, silver nanoparticles, colloidal silver, etc.), gallium containing compounds (e.g., gallium ions, gallium nitrate, gallium nanoparticles, colloidal gallium, etc.), and antimicrobial peptides. Examples of antifungals include, without limitation, amphotericin B, pyrimethamine, thiazoles, allylamines, flucytosine, caspofungin acetate, fluconazole, griseofulvin, terbinafine, amorolfine, imidazoles, triazoles (e.g., voriconazole), flutrimazole, cilofungin, echinocandines, pneumocandin omoconazole terconazole, nystatin, natamycin, griseofulvin, ciclopirox, naftifine, and itraconazole. In a particular embodiment, the antimicrobial is an antibiotic. In a particular embodiment, the antimicrobial is an antimicrobial peptide. In a particular embodiment, the nanofiber and/or nanofiber structure comprises an antimicrobial peptide and at least one other antimicrobial (e.g., antibiotic). Antimicrobial peptides may be therapeutically effective against one or more bacteria. Examples of antimicrobial peptides are provided in the Antimicrobial Peptide Database (aps.unmc.edu/AP/main.php). Examples of antimicrobial peptides are also disclosed in U.S. Pat. Nos. 7,465,784, 9,580,472, 10,144,767, U.S. Patent Application Publication No. 20090156499, U.S. Patent Application Publication No. 20150259382, U.S. Patent Application Publication No. 20140303069, and PCT/US2019/039792, each incorporated by reference herein. In a particular embodiment, the antimicrobial peptide has fewer than about 50 amino acids, fewer than about 25 amino acids, fewer than about 20 amino acids, fewer than about 17 amino acids, fewer than about 15 amino acids, fewer than 12 amino acids, fewer than 10 amino acids, or fewer than 9 amino acids. In a particular embodiment, the antimicrobial peptide has more than about 6 amino acids, particularly more than about 7 amino acids.

The antimicrobial peptide may have capping, protecting and/or stabilizing moieties at the C-terminus and/or N-terminus. Such moieties are well known in the art and include, without limitation, amidation and acetylation. In a particular embodiment, the peptides of the instant invention are amidated. The peptide template may also be lipidated or glycosylated at any amino acid (i.e., a glycopeptide). In particular, these peptides may be PEGylated to improve druggability. The number of the PEG units (NH₂(CH₂CH₂O)CH₂CH₂CO) may vary, for example, from 1 to about 50.

The antimicrobial peptide may also comprise at least one D-amino acid instead of the native L-amino acid. The antimicrobial peptide may comprise only D-amino acids. In a particular embodiment, the antimicrobial peptides comprise D-amino acids which are spaced apart by about 1, 2, 3, and/or 4 (e.g., 3) consecutive L-amino acids.

The antimicrobial peptides may contain at least one derivative of standard amino acids, such as, without limitation, fluorinated residues or nonstandard amino acids (e.g., beta-amino acids). In yet another embodiment, the peptide may also be circulated head to tail or locally involving a few residues.

In a particular embodiment of the instant invention, the peptide comprises the sequence: GX₁KRLVQRLKDX₂LRNLV (SEQ ID NO: 1), wherein X₁ and X₂ are any amino acid or derivative (e.g., analog), particularly any hydrophobic amino acids (e.g., Ile, Leu, Val, Trp, or Phe). In a particular embodiment, the Leu at positions 5, 9, and 13 are D-amino acids. In a particular embodiment, either one or both of the Leu at positions 5 and 9 are substituted with isoleucine (e.g., D-Ile). X₁ and X₂ may be a hydrophobic amino acid (inclusive of derivatives and analogs) that is larger than phenylalanine and/or more hydrophobic than phenylalanine. In a particular embodiment, X₁ and X₂ are independently selected from the group consisting of phenylalanine, trifluoromethylphenylalanine, napthylalanine, biphenylalanine, methylphenylalanine, ethylphenylalanine, benzoylphenylalanine, t-butylphenylalanine, sulfomethylphenylalanine, hydroxyphenylalanine, methoxyphenylalanine, and hydroxymethylphenylalanine. The recited substituents may be located at any position of the aromatic ring of phenylalanine, particularly at position 4. In a particular embodiment, X₁ and X₂ are independently selected from the group consisting of phenylalanine, trifluoromethylphenylalanine (e.g., 4-trifluoromethylphenylalanine), napthylalanine (e.g., 2-napthylalanine), and biphenylalanine (e.g., 4-phenyl-phenylalanine). In a particular embodiment, neither X₁ nor X₂ are phenylalanine. In a particular embodiment, X₁ nor X₂ are biphenylalanine. In a particular embodiment, the antimicrobial peptide comprises 17BIPHE2 (GX₁KRLVQRLKDX₂LRNLV, wherein X₁ and X₂ are biphenylalanines (SEQ ID NO: 2)).

In a particular embodiment, the antimicrobial peptide comprises the sequence: WWWX₁X₂X₃X₄W (SEQ ID NO: 3), wherein X₁-X₄ are any amino acid or derivative thereof (e.g., an analog). In a particular embodiment, X₁ is selected from the group consisting of Arg, Ile, Leu, and Val. In a particular embodiment, X₁ is Leu. In a particular embodiment, X₂ and X₃ are positively charged amino acids (e.g., selected from the group of Arg, Lys, His, and a derivatives or analogs thereof). In a particular embodiment, X₂ and X₃ are independently Arg or Lys. In a particular embodiment, X₂ and X₃ are independently Arg. In a particular embodiment, X₄ is selected from the group consisting of Ile, Arg, and Lys. In a particular embodiment, X₄ is Arg.

In a particular embodiment, the antimicrobial peptide comprises the sequence: RRRWWW (SEQ ID NO: 4), RRRWWWW (SEQ ID NO: 5), RRRWWWWX (SEQ ID NO: 6), or wherein X is any amino acid or derivative thereof (e.g., an analog) (e.g., alanine, isoleucine, leucine, methionine, phenylalanine, valine, proline, or glycine; particularly alanine, isoleucine, leucine, valine, or glycine; particularly leucine, valine, or alanine). In a particular embodiment, the peptide comprises RRRWWWWV (SEQ ID NO: 7). In a particular embodiment, the antimicrobial peptide comprises the sequence: X₁X₂X₃WWWWX₄ (SEQ ID NO: 8), wherein X₁, X₂, X₃, and X₄ are independently any amino acid or derivative thereof (e.g., an analog). In a particular embodiment, X₄ is alanine, isoleucine, leucine, methionine, phenylalanine, valine, proline, or glycine; particularly alanine, isoleucine, leucine, valine, or glycine; particularly leucine, valine, or alanine. In a particular embodiment, X₁, X₂, and X₃ are independently lysine or its analogs (e.g., sidechain-shortened analogs such as ornithines), arginine or its analogs, histidine or its analogs, serine, proline, glycine, aspartate, or glutamate. In a particular embodiment, X₁, X₂, and X₃ are independently lysine or its analogs (e.g., sidechain-shortened analogs such as ornithines), arginine or its analogs, or histidine or its analogs. The instant invention also encompasses analogs of the above peptides (e.g., SEQ ID NOs: 4-8) wherein one or more of the tryptophan (e.g., 1, 2, 3, or 4) are replaced with a tryptophan analog or one or more tryptophan analogs (e.g., 1, 2, 3, or 4) are inserted into the tryptophan cluster. Examples of tryptophan analogs include, without limitation, β-(1-Naphthyl)alanine, 7-azatryptophan, methyl-tryptophan (e.g., 4 or 5-methyl-tryptophan), methoxy-tryptophan (e.g., 5-methoxy-tryptophan), hydroxy-tryptophan (e.g., 5-hydroxy-tryptophan), and halo-tryptophan (e.g., 5-halo-tryptophan; e.g., halo=F, Cl, or Br).

In addition to the antimicrobial, the nanofiber and/or nanofiber structures of the instant invention may further comprise or encapsulate at least one other therapeutic agent or drug. The other therapeutic agent or drug may be, for example, within the core of the nanofiber or part of a coating on the nanofibers. In a particular embodiment, the drug or therapeutic agent is an analgesic, growth factor, anti-inflammatory, signaling molecule, cytokine, blood clotting agent, factor, or protein, pain medications (e.g., anesthetics), etc. In a particular embodiment, the drug or therapeutic agent enhances tissue regeneration, tissue growth, and wound healing (e.g., growth factors). In a particular embodiment, the drug or therapeutic agent enhances wound healing and/or enhances tissue regeneration (e.g., bone, tendon, cartilage, skin, nerve, and/or blood vessel). Such agents include, for example, growth factors, cytokines, chemokines, immunomodulating compounds, and small molecules. Growth factors include, without limitation: platelet derived growth factors (PDGF), vascular endothelial growth factors (VEGF), epidermal growth factors (EGF), fibroblast growth factors (FGF; e.g., basic fibroblast growth factor (bFGF)), insulin-like growth factors (IGF-1 and/or IGF-2), bone morphogenetic proteins (e.g., BMP-2, BMP-7, BMP-12, BMP-9; particularly BMP-2 fragments, peptides, and/or analogs thereof), transforming growth factors (e.g., TGFβ, TGFβ3), nerve growth factors (NGF), neurotrophic factors, stromal derived factor-1 (SDF-1), granulocyte-macrophage colony-stimulating factor (GM-CSF), granulocyte-colony stimulating factor (G-CSF), erythropoietin (EPO), glial cell-derived neurotrophic factors (GDNF), hepatocyte growth factors (HGF), keratinocyte growth factors (KGF), and/or growth factor mimicking peptides (e.g., VEGF mimicking peptides). Chemokines include, without limitation: CCL21, CCL22, CCL2, CCL3, CCL5, CCL7, CCL8, CCL13, CCL17, CXCL9, CXCL10, and CXCL11. Cytokines include without limitation IL-2 subfamily cytokines, interferon subfamily cytokines, IL-10 subfamily cytokines, IL-1, I-18, IL-17, tumor necrosis factor, and transforming-growth factor beta superfamily cytokines. Examples of small molecule drugs/therapeutic agents include, without limitation, simvastatin, kartogenin, retinoic acid, paclitaxel, vitamins (e.g., vitamin D3), etc. In a particular embodiment, the drug or therapeutic agent is a blood clotting factor such as thrombin or fibrinogen. In a particular embodiment, the drug or therapeutic agent is a bone morphogenetic protein (e.g., BMP-2, BMP-12, BMP-9; particularly human; particularly BMP-2 fragments, peptides, and/or analogs thereof). In a particular embodiment, the agent is a BMP-2 peptide such as KIPKASSVPTELSAISTLYL (SEQ ID NO: 9). In a particular embodiment, the agent is a BMP-2 fragment (e.g., up to about 25, about 30, about 35, about 40, about 45, about 50 amino acids, or more of BMP-2) comprising the knuckle epitope (e.g., amino acids 73-92 of BMP-2 or SEQ ID NO: 9). In a particular embodiment, the BMP-2 peptide is linked to a peptide of acidic amino acids (e.g., Asp and/or Glu; particularly about 3-10 or 5-10 amino acids such as E7, E8, D7, D8) and/or bisphosphonate (e.g., at the N-terminus).

In accordance with the instant invention, the nanofiber structures may further comprise microneedles. In a particular embodiment, the microneedle containing nanofiber structure is a Janus-type structure. In a particular embodiment, the structure comprises the nanofiber structure attached or fused to a plurality of microneedles. In a particular embodiment, the structure comprises the nanofiber structure attached or fused to a microneedle structure (e.g., a base and a plurality of microneedles). In a particular embodiment, the microneedles and/or microneedle structure comprises a polymer and an antimicrobial (e.g., antibiotic). In a particular embodiment, the antimicrobial (e.g., antibiotic) of the microneedles and/or microneedle structure is the same as the antimicrobial (e.g., antibiotic) in the nanofiber structure. In a particular embodiment, the antimicrobial (e.g., antibiotic) of the microneedles and/or microneedle structure is different than the antimicrobial (e.g., antibiotic) in the nanofiber structure. In a particular embodiment, the microneedles and/or microneedle structure further comprises an additional drug or therapeutic agent as described hereinabove for the nanofiber structure. In a particular embodiment, the polymer of the microneedles and/or microneedle structure is a hydrophilic polymer (e.g., as described hereinabove). In a particular embodiment, the polymer of the microneedles and/or microneedle structure is dissolvable and/or biodegradable. In a particular embodiment, the polymer of the microneedles and/or microneedle structure is FDA approved. In a particular embodiment, the polymer is polyvinylpyrrolidone (PVP). In a particular embodiment, the polymer is PVA. In a particular embodiment, the polymer is PLGA. The microneedles and/or microneedle structure may also be synthesized within a mold or micromold (e.g., a metal, plastic, silicone, or other material) to assist in the formation of the desired shape.

In a particular embodiment, the microneedles have a height (e.g., average height) of less than about 5 mm, less than about 1 mm, less than about 900 μm, less than about 800 μm, less than about 700 μm, less than about 600 μm, less than about 500 μm, or less than about 400 μm. In a particular embodiment, the microneedles have a height (e.g., average height) of between 50 μm and 5 mm, 1 mm, 900 μm, 800 μm, 700 μm, 600 μm, 500 μm, or 400 μm. In a particular embodiment, the microneedles have a height (e.g., average height) of between about 200 μm and 400 μm. In a particular embodiment, the microneedles have a base broader or wider than the tip. In a particular embodiment, the microneedles are conical or pyramidal in shape. In a particular embodiment, the microneedles have a base diameter or width that is equal to or less than the height of the microneedle.

The microneedles and/or microneedle structure may comprise a high density of microneedles. In a particular embodiment, the average distance (gap) between microneedles is less than about 500 μm, about 400 μm, about 350 μm, about 300 μm, about 250 μm, about 200 μm, about 150 μm, or about 100 μm. In a particular embodiment, the average distance between microneedles is between about 50 μm and 500 μm, particularly between about 50 μm and 400 μm, about 50 μm and 350 μm, about 50 μm and 300 μm, about 50 μm and 250 μm, about 50 μm and 200 μm, about 50 μm and 150 μm, or about 50 μm and 100 μm.

While the structures described above comprise the microneedles and/or microneedle structure attached to the nanofiber structure, the instant invention also encompass structures wherein the microneedles and/or microneedle structure is attached or fused to other types of scaffolds such as collagen or gelatin sponges.

Compositions comprising the nanofiber structures of the instant invention and, optionally, at least one pharmaceutically or biologically acceptable carrier are also encompassed by the instant invention.

The instant application also encompasses methods of synthesizing the nanofiber structures of the instant invention. The method comprises co-axial electrospinning a first solution comprising an amphiphilic polymer (e.g., amphiphilic block copolymer) and the antimicrobial/antibiotic (e.g., antimicrobial peptide) and a second solution comprises a hydrophobic polymer. The first and second solutions can be modifies as described hereinabove. The method may further comprise additional steps such as adding (e.g., by electrospraying) a coating to the electrospun nanofibers as described above or modifying the electrospun nanofibers as described above. In a particular embodiment, the method comprises adding or fusing a microneedle structure to the electrospun nanofiber structure as described above.

In accordance with the instant invention, the nanofiber structures may be used in inducing and/or improving/enhancing wound healing and/or treating, inhibiting, and/or preventing a microbial infection (e.g., bacterial infection (e.g., comprising a bacterial biofilm) or fungal infection). In a particular embodiment, the wound comprises a bacterial biofilm or is susceptible to bacterial biofilm growth. In a particular embodiment, the wound comprises a fungal infection. The nanofiber structures of the present invention can be used for the treatment, inhibition, and/or prevention of any injury or wound. For example, the nanofiber structures can be used to induce, improve, or enhance wound healing associated with surgery (including non-elective (e.g., emergency) surgical procedures or elective surgical procedures). Elective surgical procedures include, without limitation: liver resection, partial nephrectomy, cholecystectomy, vascular suture line reinforcement and neurosurgical procedures. Non-elective surgical procedures include, without limitation: severe epistaxis, splenic injury, liver fracture, cavitary wounds, minor cuts, punctures, gunshot wounds, and shrapnel wounds.

In accordance with the instant invention, methods for inducing and/or improving/enhancing wound healing and/or treating, inhibiting, and/or preventing a microbial or bacterial infection (e.g., comprising a bacterial biofilm) in a subject are also provided. In a particular embodiment, the wound comprises a bacterial biofilm or is susceptible to bacterial biofilm growth. In a particular embodiment, the wound is an injury to subject (e.g., such as caused by a cut, blow, or other physical impact (e.g., with an object), typically one in which the skin is cut or broken. In a particular embodiment, the wound is selected from the group consisting of diabetic ulcers or wounds, venous or arterial ulcers, injuries, or wounds, pressure ulcers, bacterial infections (e.g., necrotizing infections), and chronic wounds. The methods of the instant invention comprise administering or applying a nanofiber structure of the instant invention (e.g., as part of a wound dressing) to the subject (e.g., at or in a wound). In a particular embodiment, the method comprises administering a nanofiber structure to the subject and another therapeutic agent as described hereinabove (i.e., the therapeutic agent is not contained within the nanofiber structure). When administered separately, the nanofiber structure may be administered simultaneously and/or sequentially with the additional therapeutic agent. The methods may further comprise debriding the wound.

In a particular embodiment of the instant invention, methods for modulating (increasing) hemostasis; inhibiting blood loss; and/or treating hemorrhage are provided.

In a particular embodiment, the method comprises administering the nanofiber structure (e.g., in a wound dressing) to the wound or site of bleeding. In a particular embodiment, the nanofiber structure further comprises a blood clotting factor such as thrombin and/or fibrinogen.

The methods may comprise the administration of one or more nanofiber structures. For example, the method may comprise removing an applied nanofiber structure from the subject and applying or administering another nanofiber structure. The removal and application of a nanofiber structure may occur multiple times (e.g., as needed) and may be performed with debriding (although it is preferred to avoid debriding). For example, the method may comprise removing an applied nanofiber structure from the subject and applying or administering another nanofiber structure on a daily basis (e.g., as needed), or less frequently. The treatment method may be performed, for example, for 1, 2, 3, 4, 5, 6, 7, 8, 9, 10, 11, 12, 13, 14 days or more.

### Definitions

The singular forms “a,” “an,” and “the” include plural referents unless the context clearly dictates otherwise.

As used herein, the term “electrospinning” refers to the production of fibers (i.e., electrospun fibers), particularly micro- or nano-sized fibers, from a solution or melt using interactions between fluid dynamics and charged surfaces (e.g., by streaming a solution or melt through an orifice in response to an electric field). Forms of electrospun nanofibers include, without limitation, branched nanofibers, tubes, ribbons and split nanofibers, nanofiber yarns, surface-coated nanofibers (e.g., with carbon, metals, etc.), nanofibers produced in a vacuum, and the like. The production of electrospun fibers is described, for example, in Gibson et al. (1999) AlChE J., 45:190-195.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., polysorbate 80), emulsifier, buffer (e.g., TrisHCl, acetate, phosphate), water, aqueous solutions, oils, bulking substance (e.g., lactose, mannitol), excipient, auxiliary agent or vehicle with which an active agent of the present invention is administered. Suitable pharmaceutical carriers are described in “Remington's Pharmaceutical Sciences” by E. W. Martin (Mack Publishing Co., Easton, Pa.); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients (3rd Ed.), American Pharmaceutical Association, Washington.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

“Hydrophobic” designates a preference for apolar environments (e.g., a hydrophobic substance or moiety is more readily dissolved in or wetted by non-polar solvents, such as hydrocarbons, than by water). In a particular embodiment, hydrophobic polymers may have aqueous solubility less than about 1% wt. at 37° C. In a particular embodiment, polymers that at 1% solution in bi-distilled water have a cloud point below about 37° C., particularly below about 34° C., may be considered hydrophobic.

As used herein, the term “hydrophilic” means the ability to dissolve in water. In a particular embodiment, polymers that at 1% solution in bi-distilled water have a cloud point above about 37° C., particularly above about 40° C., may be considered hydrophilic.

As used herein, the term “amphiphilic” means the ability to dissolve in both water and lipids/apolar environments. Typically, an amphiphilic compound comprises a hydrophilic portion and a hydrophobic portion.

The term “antimicrobials” as used herein indicates a substance that kills or inhibits the growth of microorganisms such as bacteria, fungi, viruses, or protozoans.

As used herein, the term “antiviral” refers to a substance that destroys a virus and/or suppresses replication (reproduction) of the virus. For example, an antiviral may inhibit and or prevent: production of viral particles, maturation of viral particles, viral attachment, viral uptake into cells, viral assembly, viral release/budding, viral integration, etc.

As used herein, the term “antibiotic” refers to antibacterial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, an “anti-inflammatory agent” refers to compounds for the treatment or inhibition of inflammation. Anti-inflammatory agents include, without limitation, non-steroidal anti-inflammatory drugs (NSAIDs; e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, methotrexate, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), rapamycin, acetaminophen, glucocorticoids, steroids, beta-agonists, anticholinergic agents, methyl xanthines, gold injections (e.g., sodium aurothiomalate), sulphasalazine, and dapsone.

As used herein, the term “subject” refers to an animal, particularly a mammal, particularly a human.

As used herein, the term “prevent” refers to the prophylactic treatment of a subject who is at risk of developing a condition resulting in a decrease in the probability that the subject will develop the condition.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “analgesic” refers to an agent that lessens, alleviates, reduces, relieves, or extinguishes pain in an area of a subject's body (i.e., an analgesic has the ability to reduce or eliminate pain and/or the perception of pain).

As used herein, the term “small molecule” refers to a substance or compound that has a relatively low molecular weight (e.g., less than 2,000). Typically, small molecules are organic, but are not proteins, polypeptides, or nucleic acids.

The term “hydrogel” refers to a water-swellable, insoluble polymeric matrix (e.g., hydrophilic polymers) comprising a network of macromolecules, optionally crosslinked, that can absorb water to form a gel.

The term “crosslink” refers to a bond or chain of atoms attached between and linking two different molecules (e.g., polymer chains). The term “crosslinker” refers to a molecule capable of forming a covalent linkage between compounds. A “photocrosslinker” refers to a molecule capable of forming a covalent linkage between compounds after photoinduction (e.g., exposure to electromagnetic radiation in the visible and near-visible range). Crosslinkers are well known in the art (e.g., formaldehyde, paraformaldehyde, acetaldehyde, glutaraldehyde, etc.). The crosslinker may be a bifunctional, trifunctional, or multifunctional crosslinking reagent.

The term “wound dressing” refers to a material that is used to cover at least part of or all of a wound. A wound dressing is a dressing for application, particularly topical application, to a wound. A wound dressing is generally placed proximal to or on a wound and possesses absorbent, adhesive, and/or protective properties. Wound dressings may be in direct or indirect contact with a wound.

The following examples illustrate certain embodiments of the invention. They are not intended to limit the invention in any way.

### EXAMPLE 1

### Materials and Methods

### Materials

Poly(ε-caprolactone) (PCL) (Mw=70˜90 kDa) and Pluronic® F127 were bought from Sigma-Aldrich (St. Louis, Mo.). Dulbecco's Modified Eagle Media (DMEM) and RPMI 1640 media were bought from Thermo Fisher Scientific Gibco (Waltham, Mass.). Dichloromethane (DCM) and N,N-dimethylformamide (DMF) were acquired from Thermo Fisher Scientific (Waltham, Mass.). The 17BIPHE2 antimicrobial peptide (GX₁KRLVQRLKDX₂LRNLV, wherein X₁ and X₂ are biphenylalanines (SEQ ID NO: 2)) was prepared according to protocol (Wang, et al. (2014) ACS Chem. Biol., 9(9):1997-2002). Methicillin-resistant Staphylococcus aureus (MRSA) USA 300 LAC, Klebsiella pneumonia ATCC13883, Acinetobacter baumannii B2367-12 and Pseudomonas aeruginosa PAO1 were obtained from American Type Culture Collection (ATCC). Columbia CAN w/5% sheep blood agar medium was purchased from Remel (Lenexa, Kans.) and Tryptic Soy Broth (TSB) bacterial medium was purchased from Thermo Fisher Scientific Oxoid (Waltham, Mass.). LIVE/DEAD® BacLight™ bacterial viability kit and alamarBlue® cell viability assay kit was purchased from Thermo Fisher Scientific Invitrogen (Waltham, Mass.).

### Fabrication of Molecularly Engineered Peptides-Loaded Nanofibers

A co-axial electrospinning setup was used to encapsulate peptides in the core of Pluronic® F127/17BIPHE2-PCL core-shell nanofibers following protocol (Jiang, et al.

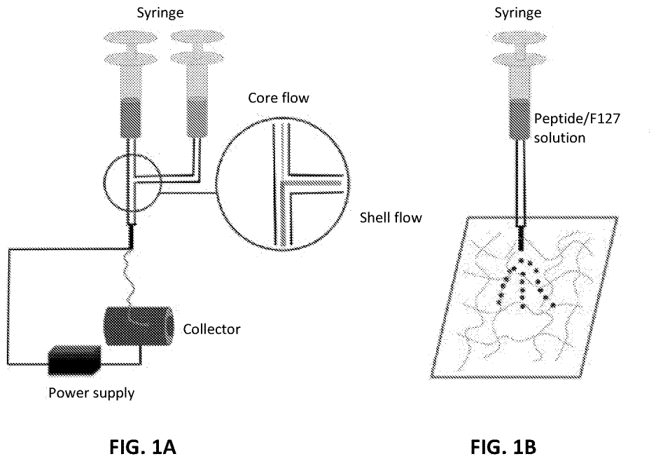

(2015) Pharm. Res., 32(9):2851-2862). Briefly, PCL was dissolved in a solvent mixture consisting of DCM and DMF with a ratio of 4:1(v/v) at a concentration of 10% (PCL) (w/v). To prepare F127-PCL core-shell fibers, 1 g of Pluronic® F127 was dissolved in ddH₂O to form the aqueous phase. To prepare F127/17BIPHE2-PCL core-shell fibers, one gram Pluronic® F127 and 25 mg 17BIPHE2 were dissolved in ddH₂O. The polymer phase was pumped at a flow rate of 0.5 ml/hour and the aqueous phase was pumped at a flow rate of 0.02 ml/hour while a potential of 20 kV was applied between the spinneret (a 22-gage needle) and a grounded collector located 12 cm apart from the spinneret. A rotating drum was used to collect membranes composed of random fibers with a rotating speed less than 100 rpm. Then, the obtained fiber samples were divided into two parts. One part was stored in 4° C. named as F127/17BIPHE2-PCL (FIG. 1A). The other part was coated with 10 mg 17BIPHE2 named as F127/17BIPHE2-PCL-S (FIG. 1B). Briefly, the F127/17BIPHE2 aqueous solution was deposited onto the fibers through electrospraying. All the fiber samples were sterilized by ethylene oxide gas prior to cell culture and in vivo animal study.

### Morphology Characterization of Nanofibers

The morphology of fiber samples was characterized by scanning electron microscopy (SEM) (FEI, Quanta 200, OR). To avoid charging, polymeric fiber samples were fixed on a metallic stud with a double-sided conductive tape and coated with platinum for 4 minutes in vacuum at a current intensity of 10 mA using a sputter coater. SEM images were acquired at an accelerating voltage of 30 kV. To confirm the core-shell structure, the FITC-labeled bovine serum albumin (BSA) was used to form F127/FITC-BSA-PCL core-shell nanofibers using the above fabrication procedure. The obtained fibers were collected on a coverslip and then imaged by a laser scanning confocal microscope (LSCM) (Zeiss, LSM 710).

### Encapsulation Efficiency and In Vitro Drug Release Study

In vitro release of 17BIPHE2 from the fibers was evaluated by immersing 10 mg fiber samples in 10 mL PBS solution at 37° C. The supernatants were collected at each time point and replaced by fresh PBS solutions. The 17BIPHE2 concentrations of all collected samples were separated on a Waters HPLC system and determined by UV-visible spectrum at 220 nm. Since only the peptide was detected, the released peptide was then measured by UV directly.

The drug encapsulation efficiency was determined as follows. A known mass of nanofiber membrane was dissolved in 1 mL of DCM, and the solution was added dropwise to 20 mL of methanol in which the polymer was precipitated and the peptide was dissolved. After centrifugation of the methanol solution, the liquid supernatant was detected by HPLC at λₘₐₓ=220 nm. The encapsulation efficiency was calculated by the following equation: encapsulation efficiency (%)=[the actual amount of drug in the sample (g)]/[the theoretical amount of drug in the sample (g)]×100%.

### In Vitro Antibacterial Efficacy Test

The antibacterial activity of F127/17BIPHE2-PCL and F127/17BIPHE2-PCL-S core-shell nanofibers was investigated. Single bacterial colonies of methicillin-resistant S. aureus (MRSA, USA 300), K. pneumoniae, A. baumannii and P. aeruginosa were picked up by inoculating loops and cultured at 37° C. and 200 rpm in liquid TSB overnight. Ten μL bacterial culture was added into 2 mL fresh TSB and incubated for additional ˜2 hours. Then, the cultures were centrifuged and washed with PBS twice. Bacteria were re-suspended and then diluted into 1.0×10⁶ CFU/mL in PBS. One mg of PCL or Pluronic® F127/17BIPHE2-PCL core-shell nanofiber membranes was co-incubated with the bacteria solution for 2 hours at 37° C. Total living bacteria were determined by culturing on agar plates. Log reduction of bacteria was calculated by the following equation: log reduction=log(cell count of control)−log(survivor count in peptide treatment group).

### In Vitro Cytotoxicity Test

The in vitro cytotoxicity of nanofiber membranes to skin cells and monocytes was investigated by determining the cell viability of co-incubated HaCaT cells (human keratinocyte cell line) and U937 cells (macrophage/monocyte cells) as described (Su, et al. (2017) Adv. Healthcare Mater., 6(6):1601173). Nanofiber membranes were firstly sterilized by ethylene oxide. HaCaT cells were cultured in DMEM with 10% FBS. U937 cells were cultured in RPMI 1640 with 10% FBS. HaCat and U937 cells were seeded in 24-well plates. Each well contains 2.5×10⁴ cells and 1 mL culture media. The pre-sterilized slides were placed into the wells with the surface coatings contacting with the cells. The plate containing cells and slides was cultured for 5 days and the culture medium was refreshed every 2 days. On days 1, 3, and 5, the cell viability was investigated by alamarBlue® assay. The cells were also stained with the LIVE/DEAD® kit (Invitrogen, Thermo-Fisher) by following the instructions of the manufacturer, and observed by an inverted fluorescence microscope (IX53, Olympus, Japan).

### In Vivo Antibiofilm Efficacy Test

To evaluate the antibacterial and antibiofilm efficacy in vivo, a biofilm-containing chronic wound model was established (Brackman, et al. (2013) J. Appl. Microbiol., 114(6):1833-1842). Briefly, MRSA was grown in TSB overnight. Subsequently, 100 μL bacterial strain was pipetted into 4 mL fresh TSB medium for 3 hours followed by PBS washing for three times. Then, the bacterial concentration was adjusted to 1×10⁸ CFU/mL and stored in the ice box before use. One hundred female 005314-TALLYHO/JngJ diabetic defective mice (10-11 weeks, 30-35 g, GLU>200 mg/dL) fed with standard pellet diet and water were used.

The biofilm was established in 005314-TALLYHO/JngJ mice excisional wounds. Two 6 mm-diameter full thickness wounds were created on the back of a mouse using a disposable biopsy punch (Integra Miltex®, Kai Medical, USA) and fixed with a wound splint (Grace Bio-labs, Inc, Bend, Oreg.). The wounds were all inoculated with 10 μL of 1×10⁸ CFU/mL MRSA instantly after surgery, and mupirocin 2% was applied to treat the wounds at day 2 (24 hours after surgery). Then, the surrounding and wound tissue was collected using an 8 mm-diameter punch and the biofilm was confirmed by CFU count, LIVE/DEAD® staining and SEM observation.

The biofilm formation was performed as above-mentioned. The mice were then divided into control groups and peptide groups. Control groups' wounds were treated with 6-mm diameter F127-PCL core-shell nanofiber discs alone. The peptide groups' wounds were treated with 6-mm diameter F127/17BIPHE2-PCL-S core-shell nanofiber discs. In this case, the mice were divided into two groups. One group was treated with fiber dressings once during the period of 3 days. The other group was treated with fiber dressings with daily replacement for 3 and 7 days. After treatment, the mice were euthanized and the wound and surrounding tissue was collected by an 8-mm diameter punch into sterilized tubes. Then, 1 mL sterilized PBS was added in each tube, which was blended by a homogenizer (Fisherband). Subsequently, the mixed liquid was diluted and plated on agar dishes. All the dishes were inoculated in a 37° C. microbial incubator for 18 hours and the CFU numbers were counted.

### Histological and Immunohistochemical Analysis

To evaluate healing and inflammation in wound areas, half of the samples collected after euthanasia were fixed with 4% paraformaldehyde for 24 hours and then embedded in paraffin and cross sectioned to 4 μm thickness slices. Partial slides were stained with Hematoxylin-Eosin (Thermofisher). Subsequently, the other half of the samples were collected and the expression of inflammatory cytokines was analyzed using a multi-analyte flow assay kit (LEGENDplex™ Mouse Inflammation Panel (13-plex) with Filter Plate), which were measured at day 3 and day 7. The experiment was performed based on the standard protocol provided by the manufacturer (Biolegend, San Diego, Calif.).

### Ex Vivo Human Skin Antibacterial Efficacy Test

F127/17BIPHE2-PCL-S core-shell nanofiber membranes and F127/PCL nanofiber membranes were cut into 8-mm diameter discs and sterilized by ethylene oxide before skin culture. The human skin tissues were collected from patients who underwent plastic surgery. After collection, skin tissues were kept on ice. All the samples were incubated with DMEM within 2 hours after surgery. Fat tissues were removed and the skin tissue was rinsed in PBS twice in order to remove blood. Then, the skin tissue was cut into 2 cm×2 cm. PCL was melt in a customized mold and form a sheet with a size of 2 cm×2 cm×0.2 cm. The tissue was fixed on PCL sheet by three or four staple clips on the corners and then placed in a 6 cm-diameter culture dish. The liquid-air-culture was applied. Approximately 7 ml DMEM medium with 10% FBS was added in each dish and the dermal layer was kept soaking in the medium with the epidermal layer exposed to air. All the cultures were maintained at 37° C. under 95% air and 5% CO₂. After 1-day incubation, a wound was generated by an 8-mm punch in the center of each skin fragment. The wound depth was around 1 mm. P. aeruginosa was prepared by the same method introduced above. Then, P. aeruginosa was diluted by sterilized PBS into 1×10⁴ CFU/ml as a bacterial inoculation liquid. Twenty μL inoculation bacterial liquid was added into wound. F127/17BIPHE2-PCL-S core-shell nanofibers membranes or PCL nanofiber discs were put on wound immediately. All the cultures were maintained at 37° C. under 95% air and 5% CO₂ for 2 hours. The fiber membranes were then removed and the wound area and wound edge of skin sample was punched off by a 10-mm punch. The skin samples were weighted and then put into 15 ml centrifuge tube followed by adding 1 ml PBS. Ten watts ultrasonication was applied to isolate bacteria from the tissue. Total living bacteria were determined by culturing on agar plates.

### Statistical Analysis

All the quantitative data are represented as mean±standard deviation. The obtained data were analyzed for statistical significance using one-way ANOVA tests and p<0.05 was considered statistically significant for all tests.

### Results

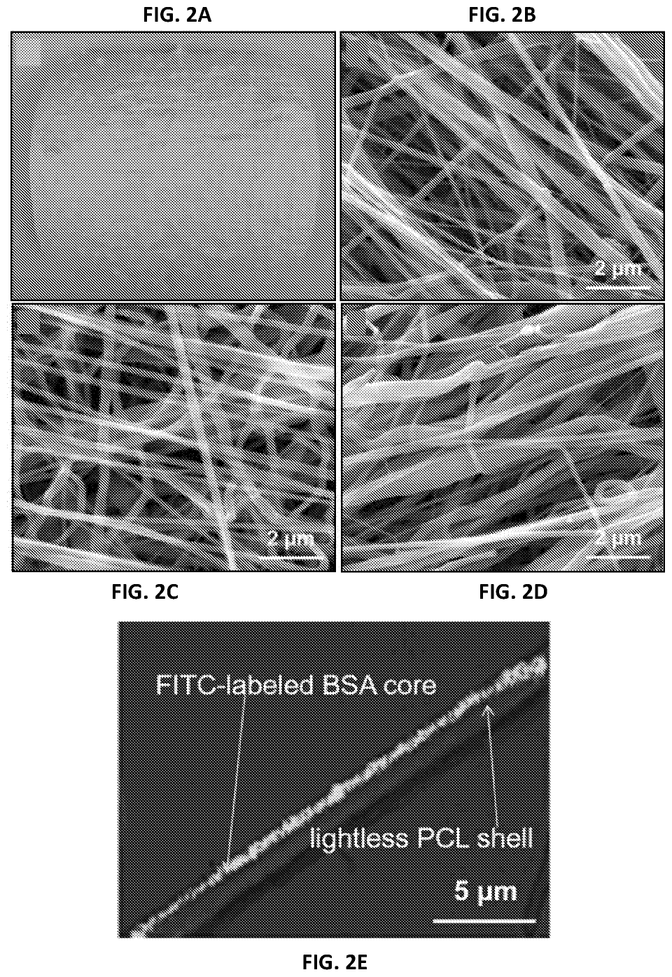

Herein, PCL was chosen as a model material because it is biocompatible and biodegradable polymers and has been approved by the FDA for certain clinical applications (Dash, et al. (2012) Mol. Pharm., 9(9):2365-2379). 17BIPHE2 was produced following a published protocol (Wang, et al. (2014) Biochim. Biophys. Acta-Biomembranes, 1838(9):2160-2172). Due to the water solubility of the peptide, co-axial electrospinning (FIG. 1) was used to encapsulate peptides to the core (Xie et al. (2012) Acta Biomnater., 8:811-819). FIG. 2 shows the macrograph and SEM images of F127-PCL, F127/17BIPHE2-PCL and F127/17BIPHE2-PCL-S samples, indicating a fibrous and porous structure. Specifically, FIG. 2A shows a photograph of F127/17BIPHE2-PCL nanofiber membranes. FIGS. 2B-2D show SEM images of F127-PCL, F127/17BIPHE2-PCL, and F127/17BIPHE2-PCL-S nanofiber samples, indicating a fibrous and porous structure. FIG. 2E shows an LSCM image of F127/FITC-BSA-PCL core-shell nanofibers, exhibiting a fluorescent core and a lightless shell, confirming the core-shell structure of the nanofibers. In general, the fibrous and porous structure mimics the extracellular matrix (ECM), and nanofiber scaffolds can serve as an artificial ECM suitable for wound healing (Smith, et al. (2004) Colloids Surf, B, 39:125-131). Moreover, the core-shell structure protects the encapsulated biological agents from a hostile microenvironment. Hence, in this study, the antimicrobial peptide retains its biological activity after encapsulation in the core-shell nanofibers (Zhang, et al. (2004) Chem. Mater., 16:3406-3409.).

The encapsulation efficiencies of 17BIPHE2 for F127/17BIPHE2-PCL and F127/17BIPHE2-PCL-S nanofiber samples were 93.5±2.8% and 89.7±3.9%, respectively. The amount of 17BIPHE2 incorporated into F127/17BIPHE2-PCL and F127/17BIPHE2-PCL-S nanofiber samples were 23.38±0.70 and 44.85±1.95 mg/g. After deposition of peptides, the encapsulation efficiency decreased probably because of the loss of some amount of the aggregated drug during the electrospray deposition.

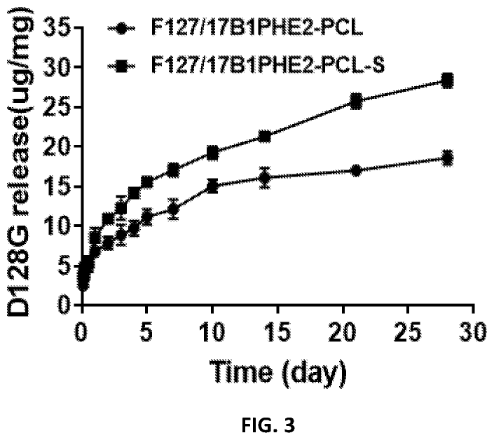

The antimicrobial peptide 17BIPHE2 can be gradually released from the fibers. Such a release was the most important factor affecting the antimicrobial activities during an extended period. To evaluate the gradual release effect of 17BIPHE2, a 28-day release profile was determined. FIG. 3 shows the in vitro release kinetics of 17BIPHE2 peptides from F127/17BIPHE2-PCL and F127/17BIPHE2-PCL-S nanofiber samples, indicating F127/17BIPHE2-PCL-S showed a higher initial burst release when compared with F127/17BIPHE2-PCL. Therefore, the initial burst from F127/17BIPHE2-PCL-S nanofiber samples allows the peptides to reach an effective concentration rapidly and the subsequent sustained release helps maintain an effective concentration for 4 weeks, thereby treating infections by a sustained release over 28 days. It is noteworthy to mention that the 17BIPHE2 deposited nanofiber membranes eluted peptides more rapidly from nanofiber formulations. It reached the peak release of non-coated fibers on day 8. The increased level of 17BIPHE2 release would result in an increase in the antibacterial efficiency. The larger burst release could provide a greater efficacy in inhibiting the bacterial growth and disrupting the biofilms. Thus, the F127/17BIPHE2-PCL-S nanofibers were selected as the wound dressing for in vivo efficacy test.

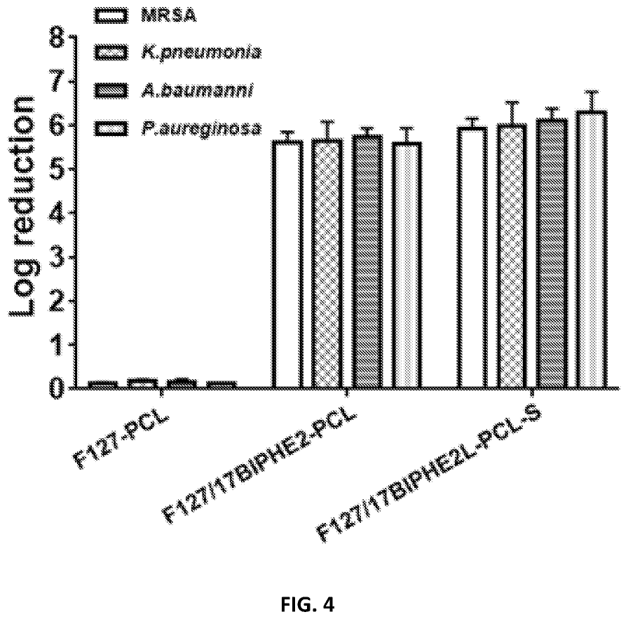

Before challenging with the biofilm, it was necessary to evaluate the in vitro antibacterial efficacy of peptide nanofiber formulations. Four pathogens including MRSA USA300, K. pneumoniae, A. baumannii and P. aeruginosa related to clinic infections were applied to determine the in vitro antibacterial activities of the antimicrobial peptides-loaded nanofiber membranes. The pathogens were cultured overnight and then re-inoculated until the bacterial growth reached the value of OD600 0.5. Then, the pathogen suspension was diluted to 1.0×10⁷ CFU/mL in PBS and co-incubated with 1 mg of F127-PCL, F127/17BIPHE2-PCL and F127/17BIPHE2-PCL-S core-shell nanofibers for 2 hours at 37° C., respectively. The bacterial Log reduction was then determined by culturing on LB agar plates. As shown in FIG. 4, the F127/17BIPHE2-PCL core-shell nanofibers effectively killed four typical infection-related bacterial strains, with 3.2, 3.6, 3.8, and 3.6-log reduction of MRSA USA300, K. pneumoniae, A. baumannii, and P. aeruginosa, respectively. Moreover, the F127/17BIPHE2-PCL-S nanofibers also effectively killed the bacteria clinical strains in vitro, with 3.4, 3.6, 3.9, and 3.7-log reduction of MRSA, K. pneumoniae, A. baumannii and P. aeruginosa, respectively (FIG. 4).

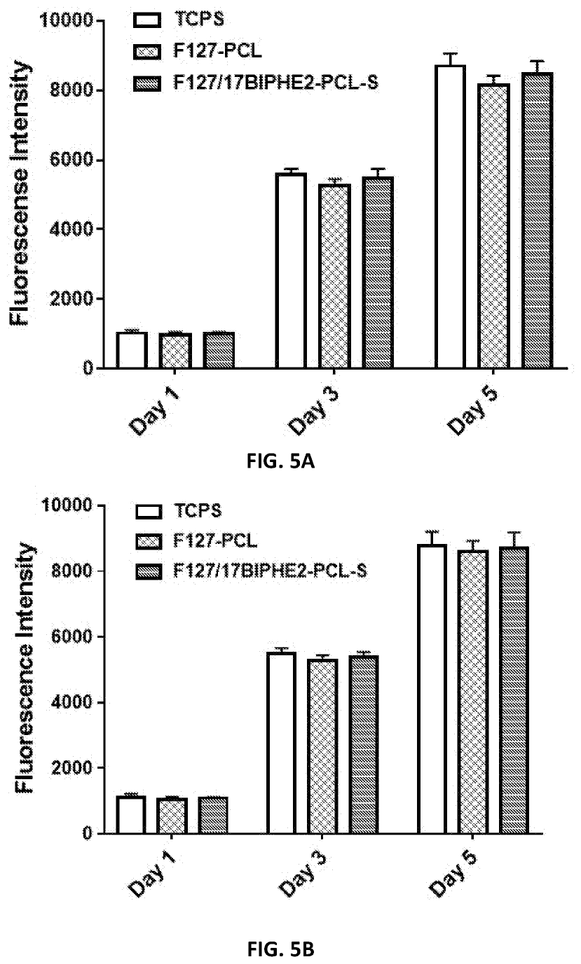

It is critical for the antimicrobial wound dressing to effectively kill bacteria (Hogue, et al. (2016) Mol. Pharm., 13(10):3578-3589). However, the cytotoxicity of the dressing should be evaluated (Archana, et al. (2015) Int. J. Biol. Macromol., 73:49-57). In order to evaluate the in vitro cytotoxicity on the skin cells and immune cells, the effect of peptide nanofiber membranes was tested on the proliferation of HaCaT and U937 cells. As shown in FIGS. 5A and 5B), F127/17BIPHE2-PCL and F127/17BIPHE2-PCL-S core-shell nanofibers membranes had no significant influences on the proliferation of HaCaT and U937 cells comparing with control group. Importantly, at this concentration (1 mg/mL), the 17BIPHE2 peptide-loaded nanofibers were able to effectively kill bacteria (FIG. 4). Overall, the cell viability results displayed no significant cytotoxicity of F127/17BIPHE2-PCL and F127/17BIPHE2-PCL-S in direct contact tests. Therefore, the antimicrobial peptides-loaded PCL nanofibers had excellent cytocompatibility allowing their application as potential trauma wound dressings.

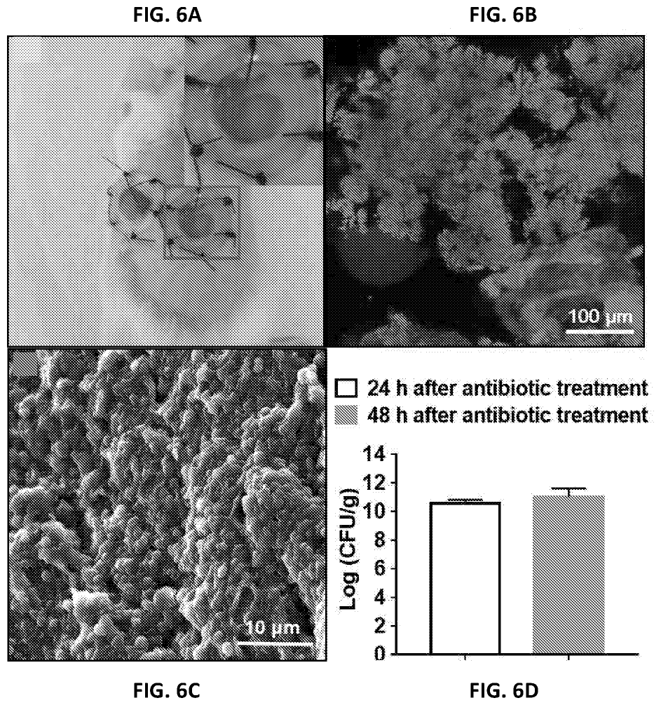

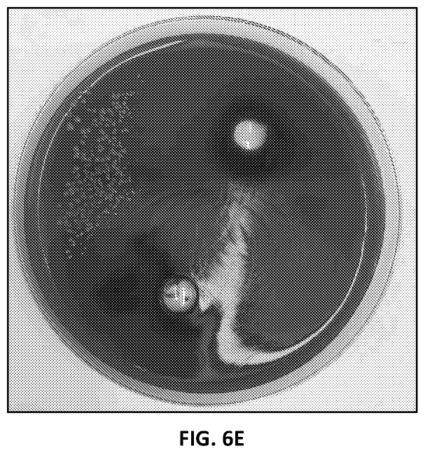

It is widely known that 99% of clinic infections are caused by bacteria in biofilms instead of planktonic ones (Gupta, et al. (2016) Arch. Microbiol., 198(1):1-15). As shown in the antibiotic agar inhibition test (FIG. 6E), the antibiotic could kill the planktonic bacteria very effectively. To further test the antibiofilm efficacy, a biofilm-containing, full-thickness excisional wound model was established in type II diabetic mice (Dash, et al. (2012) Mol. Pharm., 9(9):2365-2379; FIG. 6). FIG. 6A shows the typical wound created and fixed with splint and inoculated with MRSA for 24 hours. LIVE/DEAD® staining indicated that a huge number of bacteria were densely assembled in the tissue (FIG. 6B). SEM image also shows numerous bacteria gathered around the tissue, forming dense matrix structure, which further confirms the formation of biofilms (FIG. 6C). The CFU on the wound was also quantified. There were 10¹⁰ CFU/g bacteria resided on each surgical site and surrounding tissues (FIG. 6D). The results showed that after inoculation of 1×10⁸ CFU/mL MRSA for 24 hours and subsequent 24 hour treatment of mupirocin 2%, the biofilm was successfully formed on the wound.

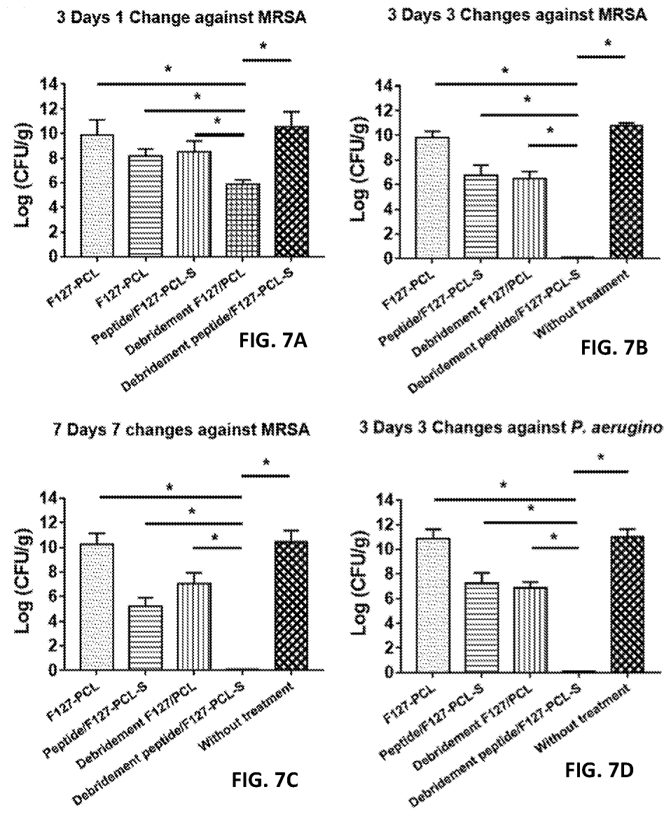

After 24 hours of bacteria inoculation and subsequent 24 hours of mupirocin 2% treatment, the mice were divided into unclean and clean groups. Unclean groups represented treatment with fiber dressings without debridement, and clean groups performed debridement (a clinical viable approach for wound infection management) before administration of fiber dressings. The F127-PCL and F127/17BIPHE2-PCL-S nanofiber dressings were applied to treat the wound for 3 and 7 days. Two different dressing change frequencies were applied during the treatment. After 3 and 7 days, all the tissue was collected and homogenized, and tissue suspension liquid was diluted to a proper concentration and plated on agar. Without debridement (the unclean group), around 1.57×10⁸ CFU/g MRSA were detected in wounds treated once with F127/17BIPHE2-PCL-S dressing for 3 days, with a 1.68-log reduction compared to the F127-PCL control group (FIG. 7). With debridement (the clean group), approximately 7.41×10⁵ CFU/g MRSA were detected in the wounds treated once with F127/17BIPHE2-PCL-S dressing for 3 days, with a 2.99-log reduction compared to the F127-PCL control group (FIG. 7).

To further improve the treatment efficacy, the dressing was changed daily (i.e. During the treatment, F127/17BIPHE2-PCL-S dressings were replaced daily). Without debridement, around 6.17×10⁶ CFU/g MRSA were detected in wounds treated with F127/17BIPHE2-PCL-S dressings daily for 3 days, with a 3.08-log reduction comparing to the F127-PCL control group (FIG. 7B). Intriguingly, with debridement, no colonies were detected in the wounds treated with F127/17BIPHE2-PCL-S dressing daily for 3 days, with a 9.86-log reduction comparing to the F127-PCL control group (FIG. 7B). In addition, after 7-day treatment, without debridement, around 1.72×10⁵ CFU/g MRSA were detected in wounds treated with F127/17BIPHE2-PCL-S dressings daily, with a 5.01-log reduction comparing to the F127-PCL control group (FIG. 7C). Similarly, with debridement, no colonies were observed in the wounds treated with F127/17BIPHE2-PCL-S dressing daily for 7 days, with a 10.24-log reduction comparing to the F127-PCL control group (FIG. 7C).

Additionally, to explore the antibiofilm activities for Gram-negative bacteria, a P. aeruginosa biofilm wound model was established, and the wound was treated with the F127/17BIPHE2-PCL-S dressing daily for 3 days. Without debridement, around 1.74×10⁷ CFU/g P. aeruginosa was detected in wounds treated with F127/17BIPHE2-PCL-S dressings daily for 3 days, with a 3.61 log reduction compared to the F127-PCL control group (FIG. 7D). Moreover, similar to the results of Gram-positive bacteria, with debridement, no colonies were detected in the wounds treated with F127/17BIPHE2-PCL-S dressing daily for 3 days, with a 10.75 log reduction compared to the F127-PCL control group (FIG. 7D).

Thus, the biofilms comprising either Gram-negative or Gram-positive bacteria on the diabetic wounds could be eliminated entirely after the combinatorial treatment of debridement and the antimicrobial peptide-loaded nanofiber dressings with daily changes for 3 and 7 days.

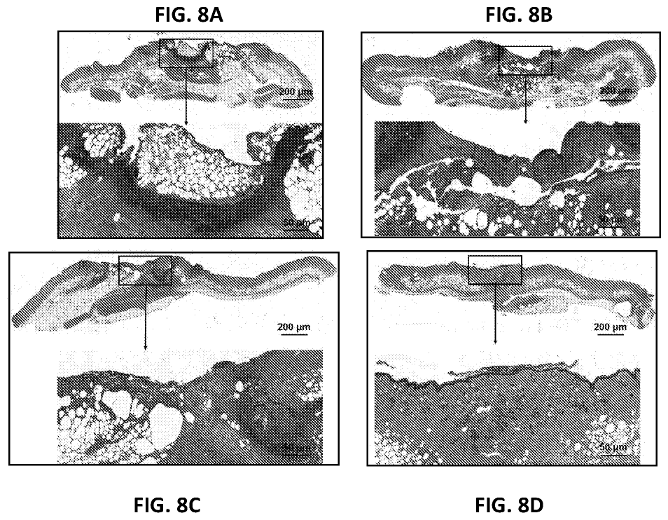

To examine the wound healing and inflammation, histology and cytokine assays were performed. As shown in FIG. 8A, the inflammatory exudates, necrosis, and granulation tissue, accompanied by acute and chronic inflammatory cell infiltration, were found in the wounds with treatment of F127-PCL nanofiber dressings. In contrast, the inflammatory exudation and inflammatory cell infiltration were reduced in the wounds with debridement and treatment of F127-PCL nanofiber dressings (FIG. 8B). The inflammatory exudation and inflammatory cell infiltration were further reduced in the wounds with treatment of F127/17BIPHE2-PCL-S nanofiber dressings (FIG. 8C). The inflammatory level was the lowest in the wounds with debridement and treatment of F127/17BIPHE2-PCL-S nanofiber dressings compared to the other three groups (FIG. 8D). In addition, new blood vessels, granulation tissues, and re-epithelialization were formed (FIG. 8D).

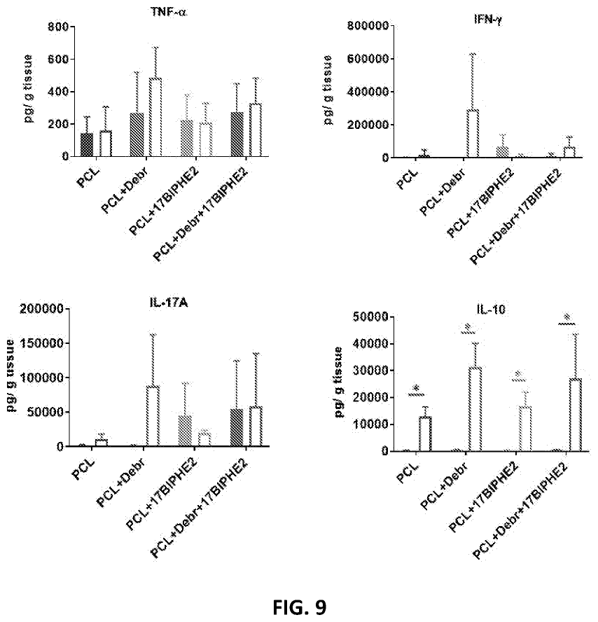

A LEGENDplex™ Mouse Inflammation Panel (13-plex) with Filter Plate kit was used to quantify the expression of cytokines including TNF-α, IFN-γ, IL-17A and IL-10. There was no significant difference between the expression levels of TNF-α, IFN-γ and IL-17A at the day 3 and day 7 (FIG. 9). However, the IL-10 expression level at day 7 was significantly higher than that at day 3 for all the groups (FIG. 9). A similar level of IL-17A and IL-10 at day 7 would suppress inflammation to avoid potential tissue damage.

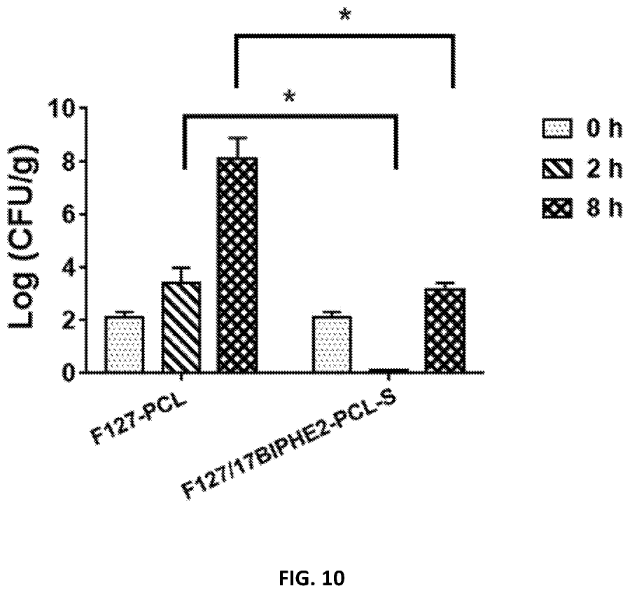

In order to further test the antibacterial efficacy of peptide-loaded nanofiber dressings, an artificial wound infection model was developed by culturing human skin tissues ex vivo. Herein, P. aeruginosa was chosen as a model bacterial for inoculation to human skin artificial wounds. A 2 cm diameter partial thickness wound was created using a surgical scalpel and inoculated with 200 CFU/mL P. aeruginosa. After administration of nanofiber dressings to the wounds, the tissues were cultured for 2 and 8 hours and the bacterial burden was quantified as shown in FIG. 10. It was seen that P. aeruginosa grew rapidly in the inoculated wounds with PCL nanofiber dressing covering. In contrast, the bacterial growth was inhibited in the inoculated wounds with 17BIPHE2-loaded PCL nanofiber dressing covering. At day 8, the CFU was reduced by 5 logs.

In summary, the successful incorporation of molecularly engineered peptides to electrospun nanofibers using co-axial electrospinning and electrospray deposition has been demonstrated. Such peptide-loaded nanofiber membranes showed an initial burst release followed by a sustained release of antimicrobial peptides for at least 28 days. The antibacterial efficacy of peptide-loaded nanofiber membranes was demonstrated against multiple antibiotic resistant bacteria in vitro, ex vivo and in vivo. These results indicate that the LL-37-engineered 17BIPHE2 retained its activity against both S. aureus and P. aeruginosa under different scenarios, confirming the usefulness of nanofibers as a peptide carrier. The co-incorporation of this peptide with conventional antibiotics can also be used for an improved antibiofilm capability (Mishra, et al. (2017) Pharmaceuticals, 10:58; Mishra, et al. (2017) Curr. Opin. Chem. Biol., 38:87-96). Thus, the present data demonstrates treatment for wounds (e.g., chronic wounds) by combining the engineered LL-37 peptide with nanofibers. Together, nanofiber membranes topically Delivering engineered peptides hold great promise in the prevention of wound infections. Electrospun nanofibers described herein can also serve as sutures, coating materials of biomedical devices, or hemostasis materials for preventing infections in different scenarios.

### EXAMPLE 2

Chronic wounds such as diabetic foot ulcers are a worldwide health problem. About 78% of chronic wounds contain biofilms, where a pathogenic bacteria community is encased in a biopolymer layer. Bacteria in biofilms are more likely to cooperate and exchange their genes resulting in much higher antibiotic resistance than planktonic bacteria. The composition and organization of biofilms limits diffusion of molecules—including antibiotics—through the structure and into the biofilm or out to the bulk fluid. Consequently, bacteria in a biofilm are refractory to host response and antibiotic treatment. Sharp debridement is currently the standard care to remove biofilms. However, vigorous and repeated debridement causes extreme discomforts to patients. This method may not be able to completely get rid of the biofilms even by removing excessive amount of tissues. The poor treatment outcomes result in high healthcare cost, amputations, a decreased quality of life, and an increased mortality. There is an urgent need to develop novel therapies for effective treatment of biofilms in chronic wounds.

Recent studies have been devoted to the development of new technologies for combating biofilms through enhancing drug diffusion and efficacy and other physical approaches. For examples, the surface of surgical implant was homogeneously coated with gold nanorods designed to efficiently convert near-infrared light into heat for elimination of attached bacteria. This approach requires the coating of gold nanorods, which is not suitable for treatment of biofilms in chronic wounds. In a different study, to improve drug diffusion and efficacy, cationic gold nanoparticles were dispersed in the biofilms and laser was used to induce vapor nanobubbles formed around plasmonic nanoparticles which can disrupt the biofilms and enhance the diffusion of drugs. This method requires the dispersion of gold nanoparticles in biofilms, which could be very difficult to realize for the biofilms in chronic wounds. The generated local heat could also cause negative effects to the tissues in the wound area. Similarly, magnetic nanoparticles were used to create artificial channels in biofilms for enhancing bacterial killing by antibiotics. This method also requires the precise control of magnetic nanoparticle movement in biofilms, which might be challenging in chronic wounds. In addition, all these studies require complicated equipment such as laser or magnet and additional training. Microneedle patches were also reported recently to treat bacterial biofilms. However, these technologies are in the early developmental stage. Most of these studies were limited to the use of traditional antibiotics which may be not effective for the treatment of antibiotic resistant bacteria. These studies only tested the in vitro efficacy against non-polymicrobial biofilms which are different from the biofilms often encountered in chronic wounds. The in vivo and ex vivo efficacy has not yet been examined. The patches cannot provide sustained release of antimicrobial agents and lack the capability in serving as scaffolds and enhancing cellular infiltration for wound healing.

Toward this end, a new approach to combating biofilms in chronic wounds was developed including the following two major aspects: i) development of novel molecularly engineered antimicrobial peptides; and ii) development of a novel system for delivery of engineered peptides. The significance of the approach is to use nature's wisdom that antimicrobial peptides remain potent for millions of years. In humans, there are several dozen defensins, but there is only one cathelicidin gene that encodes LL-37. Because this native peptide has shortcomings such as long sequence (high cost) and instability to proteases (loss of activity), LL-37 was engineered into 17BIPHE2 which is superior to the parent molecule in numerous aspects, such as antibiofilm of S. aureus USA300 and antimicrobial robustness under different salts, pH, and media conditions. Engineered peptides exhibit a broad-spectrum activity against medically significant ESKAPE pathogens. Remarkably, the engineered peptide 17BIPHE2 displays optimized protection of wax moths from S. aureus USA300 infection compared to either LL-37 or GF-17 (e.g., the native template for 17BIPHE2 engineering) and demonstrated antibiofilm activity also in a mouse model. 17BIPHE2-containing nanofiber membranes led to a five-magnitude decrease of the MRSA USA300 CFU in a biofilm-containing chronic wound model based on type 2 diabetic mice. No bacteria were observed for the biofilm-containing chronic wounds after treatment of peptide nanofiber formulations in combination with sharp debridement-a clinically viable approach for wound management.

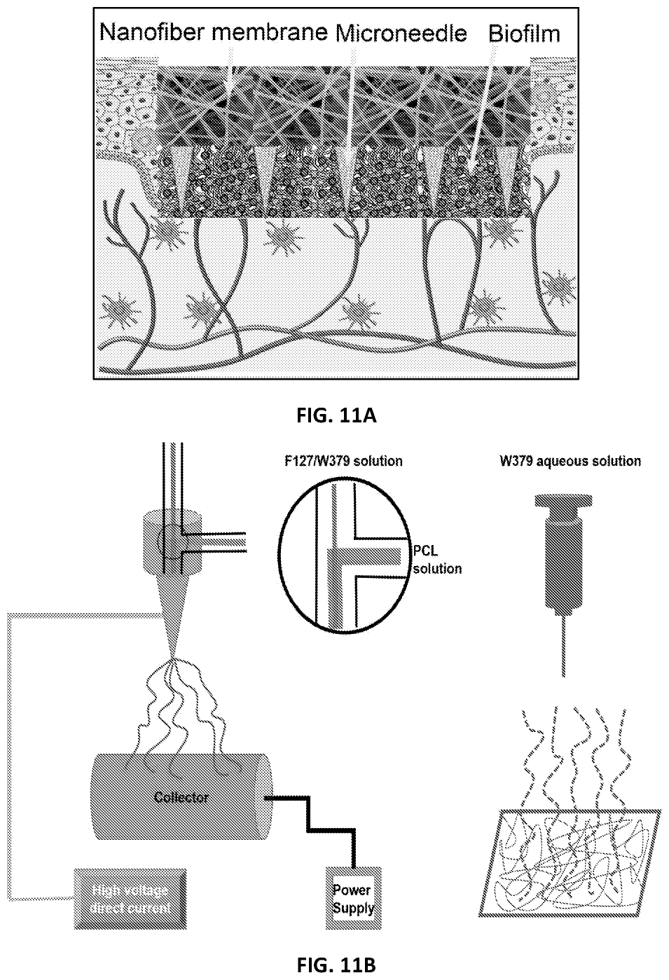

Herein, an even shorter peptide has been utilized. W379, a peptide with merely eight amino acids (RRRWWWWV; SEQ ID NO: 7), was found to have antimicrobial activity against the resistant ESKAPE pathogens, including persisters and biofilms. Microneedles have been widely used for the transdermal drug delivery as the microneedles can assist the delivery of drugs by direct penetration through the stratum corneum and/or epidermis. The dissolvable microneedle arrays containing engineered peptides can overcome the physical barrier of biofilms and penetrate into biofilms and release antimicrobial peptides from inside to kill bacteria within biofilms. Herein, a novel Janus-type antimicrobial dressing was developed by immobilizing W379 peptide incorporated microneedle arrays to the surface of peptide encapsulated nanofiber membranes for the treatment of biofilms in chronic wounds (FIG. 11A). PCL was chosen as raw materials for fabrication of nanofiber membranes because PCL has been approved by the FDA for use in other clinical applications. Polyvinylpyrrolidone (PVP) was also chosen as raw materials for fabrication of dissolvable microneedle arrays due to its water-soluble property and its FDA approval for use as an inactive ingredient.

### Materials and Methods

### Materials

Poly(ε-caprolactone) (PCL, Mw=70-90 kDa), polyvinylpyrrolidone (PVP, Mw=130 kDa) and Pluronic® F127 were bought from Sigma-Aldrich (St. Louis, Mo.). Dulbecco's modified Eagle's medium (DMEM) and RPMI 1640 medium were bought from Thermo Fisher Scientific Gibco (Waltham, Mass.). Dichloromethane (DCM) and N,N-dimethylformamide (DMF) were acquired from Thermo Fisher Scientific (Waltham, Mass.). Methicillin-resistant Staphylococcus aureus (MRSA) USA300 LAC and Acinetobacter baumannii B2367-12 were obtained from the University of Nebraska Medical Center (UNMC), while Klebsiella pneumoniae (ATCC 13883) and Pseudomonas aeruginosa PAO1 were obtained from the American Type Culture Collection (ATCC). Columbia CAN with 5% sheep blood agar medium was purchased from Remel (Lenexa, Kans.), and tryptic soy broth (TSB) bacterial medium was purchased from Thermo Fisher Scientific Oxoid (Waltham, Mass.). LIVE/DEAD® BacLight™ bacterial viability kit and alamarBlue® cell viability assay kit was purchased from Thermo Fisher Scientific Invitrogen (Waltham, Mass.).

### Fabrication of Molecularly Engineered Peptides-Loaded Nanofibers

F127/W379-PCL core-shell nanofibers which encapsulate peptides in the core of Pluronic® F127 were prepared by a co-axial electrospinning setup. Briefly, a given mass of PCL was dissolved in a solvent mixture consisting of DCM and DMF with a ratio of 4:1(v/v) at a concentration of 10% (PCL) (w/v). To prepare F127-PCL core-shell fibers, 1 g Pluronic® F127 was dissolved in 10 mL ddH₂O to form the aqueous phase. To prepare F127/W379-PCL core-shell fibers, 1 g Pluronic® F127 and 25 mg W379 were dissolved in 10 mL ddH₂O to form the aqueous phase. The polymer phase was pumped at a flow rate of 0.5 ml/hour and the aqueous phase was pumped at a flow rate of 0.02 ml/hour while a potential of 20 kV was applied between the spinneret (a 22-gage needle) and a grounded collector located 12 cm apart from the spinneret. A rotating drum was used to collect the membranes composed of random fibers with a rotating speed less than 100 rpm. Then, the obtained fiber samples were divided into two parts. One part was stored in 4° C. named as F127/W379-PCL (Scheme 1A). The other part was coated with 10 mg W379 named as F127/W379-PCL-S (Scheme 1B). Briefly, the W379 aqueous solution was deposited onto the fibers through electrospraying. All the fiber samples were sterilized by ethylene oxide gas before cell culture and in vivo animal study.

### Fabrication of Janus-Type Dressing

The fabrication of the microneedle (MN) patch was performed using a polydimethylsiloxane (PDMS) micromold (Blueacre Technology Ltd., Dundalk, Ireland) with each needle cavity being 300 μm in a round base diameter and 300 μm in height, which there were 100 (general mold) or 150 (high density mold) needles on a 6-mm circle. In brief, 50 μL of a 20 wt % PVP aqueous solution containing different concentration of W379 antimicrobial peptide, gallium nitrate, silver nitrate and traditional vancomycin was respectively deposited into the needle cavities and kept under vacuum for 10 minutes. Subsequently, a piece of F127-W379/PCL-S nanofiber was loaded onto the micromold and allowed to dry at room temperature. After complete desiccation, the Janus-type patch was detached from the silicone mold for further use. For the preparation of the control Janus-type patch, no W379 was added in the F127 or PVP solution.

### Morphology Characterization of Janus-Type Dressing

The morphology of F127-W379/PCL-S nanofiber and Janus-type samples were characterized by scanning electron microscopy (SEM) (FEI, Quanta 200, OR). To avoid charging, polymeric fiber samples were fixed on a metallic stud with a double-sided conductive tape and coated with platinum for 4 minutes in vacuum at a current intensity of 10 mA using a sputter coater. SEM images were acquired at an accelerating voltage of 30 kV.

### In Vitro Antibacterial Efficacy Test

The antibacterial activity of F127-W379/PCL-S nanofiber and Janus-type Dressing was investigated. Single bacterial colonies of MRSA, K. pneumoniae, A. baumannii, and P. aeruginosa were picked up by inoculating loops and cultured at 37° C. and 200 rpm in liquid TSB overnight. Ten microliters of bacterial cultures were added into 2 mL of fresh TSB and incubated for additional 2 hours. Then, the cultures were centrifuged and washed with PBS twice. Bacteria were resuspended and then diluted into 1.0×10⁷ CFU/mL in PBS. One milligram each of Janus-type dressing with or without W379 containing was co-incubated with the bacterial solution for 2 hours at 37° C. Total living bacteria were determined by culturing on agar plates. Log reduction of bacteria was calculated by the following equation: log reduction=log(cell count of control)−log(survivor count in peptide treatment group).

### In Vitro Cytotoxicity Test

The in vitro cytotoxicity of nanofiber membranes to skin cells and monocytes was investigated by determining the cell viability of co-incubated HaCaT cells (human keratinocyte cell line) and U937 cells as described in the previous publication. Nanofiber membranes were firstly sterilized by ethylene oxide. HaCaT cells were cultured in DMEM with 10% FBS, and U937 cells were cultured in RMPI1640 with 10% FBS. HaCat and U937 cells were seeded in 24-well plates. Each well contains 2.5×10⁴ cells and 1 mL culture media. The cells were treated by the following the procedure described in the section of cell culture and treatments. The pre-sterilized slides were placed into the wells with the surface coatings contacting with the cells. The plate containing cells and slides was cultured for 5 days and the culture medium was refreshed every 2 day. On days 1, 3, and 5, the cell viability was investigated by alamarBlue® assay.

### Ex Vivo Antibiofilm Efficacy Test

To evaluate the antibacterial and antibiofilm efficacy ex vivo, a biofilm-containing chronic wound model on human skin tissues was established. The human skin tissues were collected from patients who underwent plastic surgery. After collection, skin tissues were kept on ice. Fat tissues were removed, and the skin tissue was rinsed in PBS thrice in order to remove blood. Then, the skin tissue was cut into 2 cm×2 cm. A wound was generated by an 8 mm punch in the center of each skin fragment. The wound depth was around 1 mm. MRSA, A. baumannii, and P. aeruginosa was prepared by the same method introduced above. Twenty microliters of inoculation bacterial liquid with the concentration 1×10⁸ CFU/mL was added into the wound. All the cultures were maintained at 37° C. for 72 hours. Then, the surrounding and wound tissue was collected using a 10 mm-diameter punch, and the biofilm was confirmed by CFU count, LIVE/DEAD® staining, and SEM observation.

The biofilm formation was performed as described above. Then, F127/17BIPHE2-PCL-S core-shell nanofiber membranes or Janus-type dressings were put on the wound, different dressing changes strategies including different AMP concentration, different microneedle density and different antimicrobial agents were applied to treat the biofilm formed wound. After treatment, the wound and surrounding tissue was collected by a 10 mm-diameter punch into sterilized tubes. Then, 1 mL of sterilized PBS was added in each tube, which was blended by a homogenizer (Fisher Scientific, Hampton, N.H.). Subsequently, the mixed liquid was diluted and plated on agar dishes. All the dishes were inoculated in a 37° C. microbial incubator for 18 hours, and the CFU numbers were counted.

### In Vivo Antibiofilm Efficacy Test

A biofilm-containing chronic wound model was established. Briefly, MRSA was grown in TSB overnight. Subsequently, 100 μL of bacterial strain was pipetted into 4 mL of fresh TSB medium and cultivated for 3 hours followed by PBS washing for three times. Then, the bacterial concentration was adjusted to 1×10⁸ CFU/mL and stored in the ice box before use. Nine female 000697-B6.BKS(D)-Leprdb/J diabetic defective mice (male, 10-11 weeks, 50-55 g, GLU>200 mg/dL) fed with standard pellet diet and water were used.

The biofilm was established in 000697-B6.BKS(D)-Leprdb/J mice excisional wounds. Two 6 mm-diameter full thickness wounds were created on the back of a mouse using a disposable biopsy punch (Integra Miltex, Kai Medical, USA) and fixed with a wound splint (Grace Bio-labs, Inc., Bend, Oreg.). The wounds were all inoculated with 10 μL of 1×10⁸ CFU/mL MRSA instantly after surgery, and 2% mupirocin was applied to treat the wounds at day 2 (24 hours after surgery). Then, the surrounding and wound tissue was collected using a 10 mm-diameter punch, and the biofilm was confirmed by CFU count, LIVE/DEAD® staining, and SEM observation.

After the biofilm formation was performed, F127/17BIPHE2-PCL-S core-shell nanofiber membranes or Janus-type dressings were put on the wound, different dressing changes strategies were applied to treat the biofilm formed wound. After that, the wound and surrounding tissue was collected by a 10 mm-diameter punch into sterilized tubes. Then, 1 mL of sterilized PBS was added in each tube, which was blended by a homogenizer. Subsequently, the mixed liquid was diluted and plated on agar dishes. All the dishes were inoculated in a 37° C. microbial incubator for 18 hours, and the CFU numbers were counted.

### Statistical Analysis

All the quantitative data are represented as mean±standard deviation. The obtained data were analyzed for statistical significance using one-way ANOVA tests and *p<0.05 was considered statistically significant for all tests.

### Results

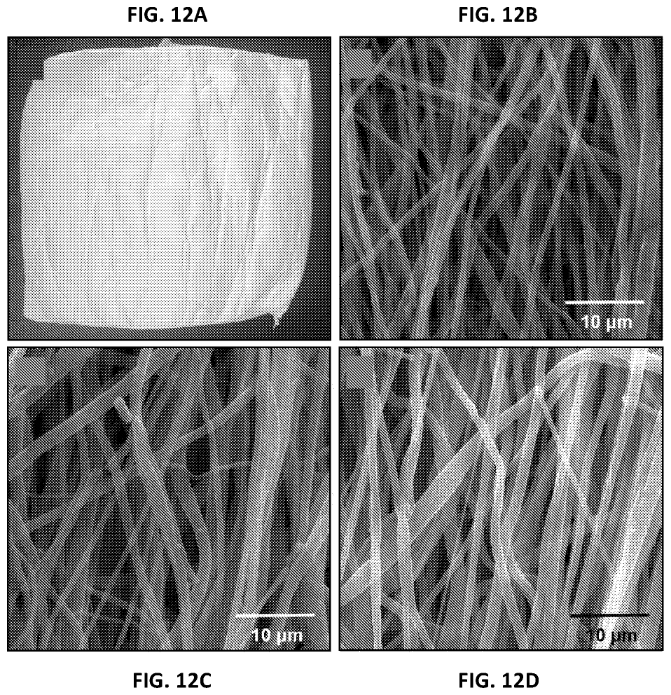

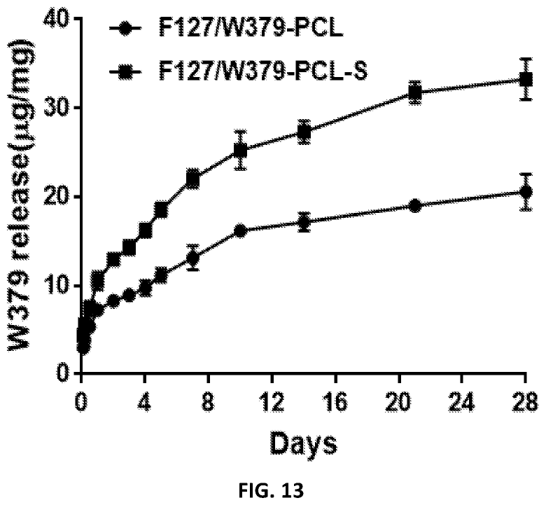

Prior to fabrication of Janus-type antimicrobial dressing, peptide-loaded nanofibers were prepared by co-axial electrospinning and electrospray deposition techniques (FIG. 11B). FIG. 12A shows a photograph of F127/W379-PCL nanofiber membrane. FIGS. 12B-12D show SEM observation images of F127-PCL, F127/W379-PCL and F127/W379-PCL-S nanofiber samples, indicating that all the samples exhibited a fibrous and porous structure. Importantly, the fibrous and porous structure can act as extracellular matrix (ECM)—mimicking scaffolds and serving as an artificial ECM suitable for wound healing. Moreover, the core-shell structure protects the encapsulated biological agents from a hostile microenvironment. The encapsulation efficiencies of W379 for F127/W379-PCL and F127/W379-PCL-S nanofiber samples were 94.3±3.3% and 90.7±2.7%, respectively. The amount of W379 incorporated into F127/W379-PCL and F127/W379-PCL-S nanofiber samples were 23.58±0.83 and 45.35±1.35 mg/g. After deposition of peptides, the encapsulation efficiency decreased, probably because of the loss of some amount of the aggregated drug during the electrospray deposition. FIG. 13 shows the in vitro release kinetics of W379 peptides from F127/W379-PCL and F127/W379-PCL-S nanofiber samples, indicating a burst release followed by a sustained release over 28 days. The sustained release of peptides can help to prevent the recurrence of infection or biofilm formation.

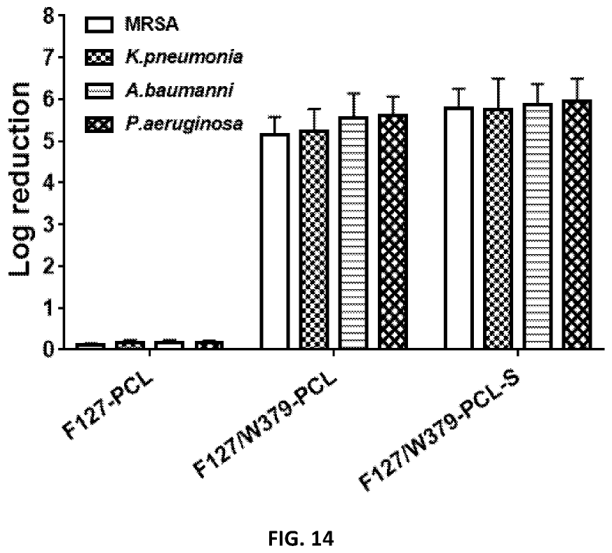

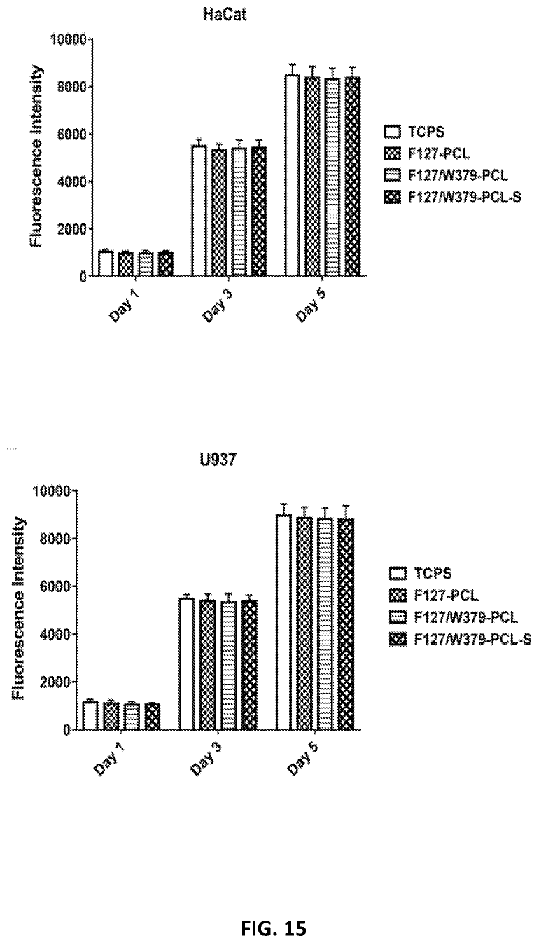

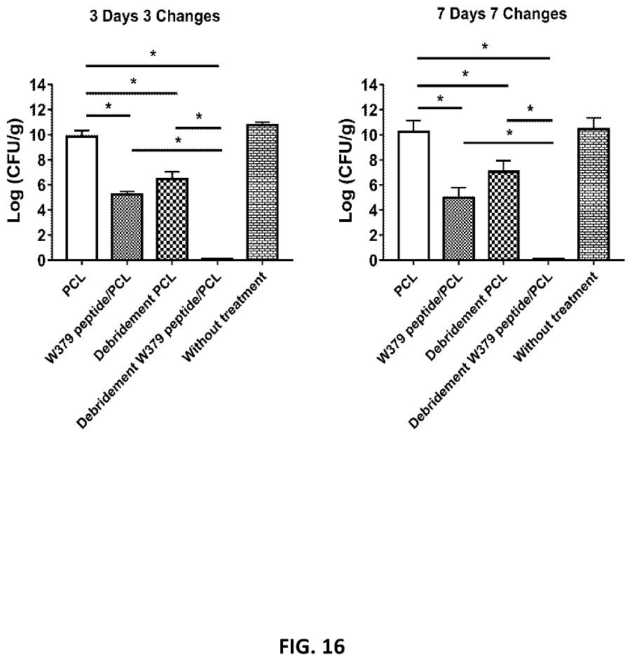

The in vitro antibacterial efficacy of peptides-loaded nanofiber membranes was evaluated. Four pathogens including MRSA, K. pneumoniae, A. baumanni, and P. aeruginosa were applied to determine the in vitro antibacterial activities of the peptides-loaded nanofiber membranes. As shown in FIG. 14, the F127/W379-PCL core-shell nanofibers effectively killed four typical infection-related bacterial strains, with 5.0, 5.1, 5.4, and 5.4-log reduction of MRSA USA300, K. pneumoniae, A. baumannii, and P. aeruginosa, respectively. Moreover, the F127/W379-PCL-S nanofibers also effectively killed the bacteria clinical strains in vitro, with 5.6, 5.5, 5.7, and 5.8-log reduction of MRSA, K. pneumoniae, A. baumannii, and P. aeruginosa, respectively (FIG. 14). Interestingly, peptides loaded PCL nanofibers (≤1 mg/ml) have no significant influences on the proliferation of skin cells, including keratinocytes and dermal fibroblasts (FIG. 15). Importantly, at this concentration (1 mg/ml), W379 peptide-loaded nanofibers were effective to kill bacteria (FIG. 14). Furthermore, the in vivo test showed about five-magnitude reduction of CFU after 3 membrane changes in 3 days (FIG. 16). In combination with debridement, no MRSA bacteria were detected after 3 membrane changes in 3 days (FIG. 16). These results indicate W379 peptides have similar antimicrobial activity as 17BIPHE2. However, the sharp debridement is necessary to combine with peptide-loaded nanofiber membranes to eradicate biofilm. Greater penetration of antimicrobial peptides into the biofilms was desired.

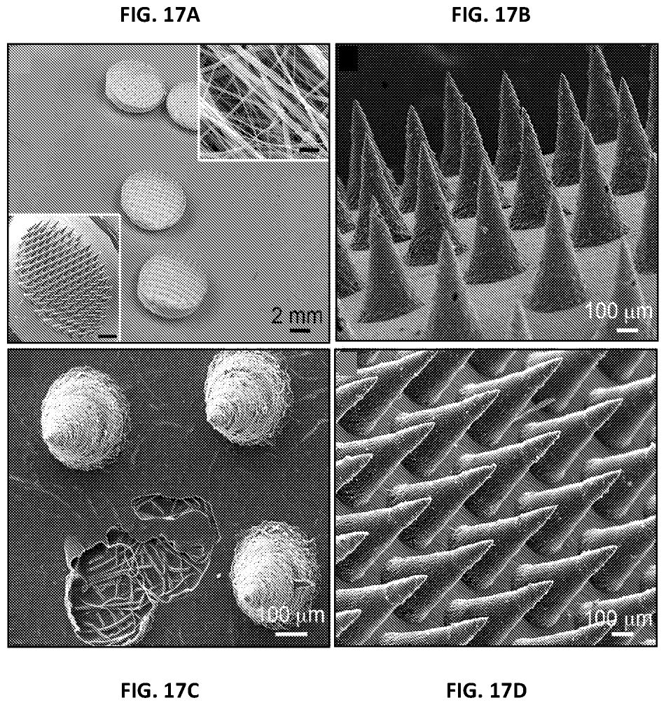

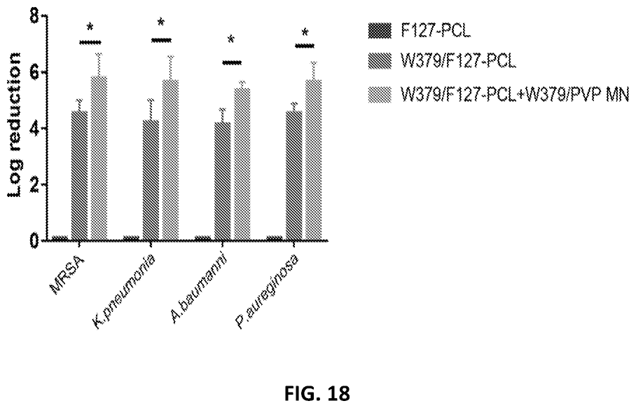

To address this, a Janus-type antimicrobial dressing concept was developed. First, a Janus-type antimicrobial dressing was fabricated (FIG. 17A). FIG. 17C shows a SEM image of the bottom substrate indicating the nanofibrous morphology. FIGS. 17B and 17D show the W379 peptide loaded PVP microneedle arrays with two different densities (100 microneedles per membrane and 150 microneedles per membrane). The in vitro antibacterial activity of Janus-type dressings was tested. Compared to F127-PCL core-shell nanofiber membranes and W379/F127-PCL core-shell nanofiber membranes, the Janus-type dressing comprising W379/F127-PCL core-shell nanofiber membranes and W379 loaded PVP microneedles showed larger Log reduction of MRSA, K. pneumoniae, A. baumannii, and P. aeruginosa (FIG. 18).

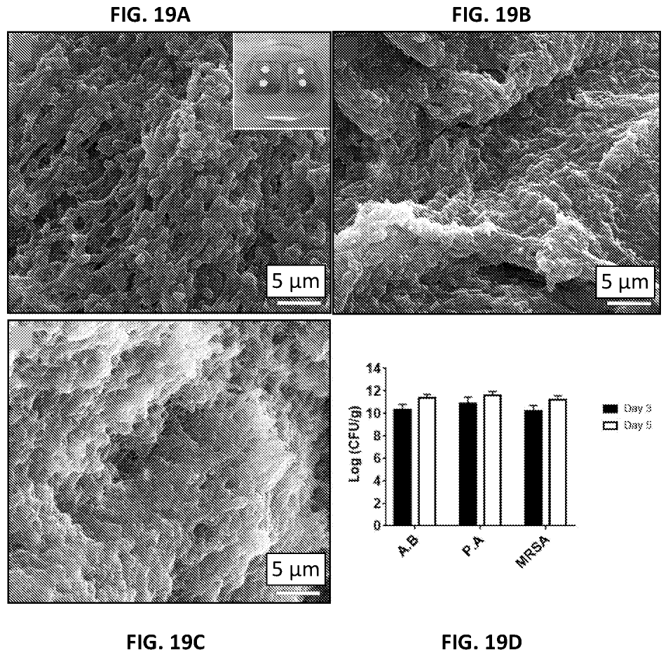

To assess the efficacy against biofilms, a biofilm-containing human skin wound model was established using different pathogens (FIG. 19). FIGS. 19A-19C show SEM images of the morphology of A. baumannii, P. aeruginosa, and MRSA USA300 biofilms on the excisional wounds in human skin explants. The CFU of different biofilms (10¹⁰-10¹² CFU/g) formed after 3 and 5 days of bacteria inoculation was quantified as shown in FIG. 19D. The efficacy of Janus-type antimicrobial dressings in eliminating biofilms in excisional wounds created in human skin explants was then tested. Different dressings were applied to the biofilm-containing wounds for 24 hours and CFU counting was performed. The treatment with PCL-F127/W379+PVP/W379 MN dressings showed the best performance (5-6 Log reduction) against A. baumannii, P. aeruginosa, and MRSA USA300 biofilms among all the treatment groups (FIG. 20A). Compared with PCL-F127/379+aqueous W379 treatment, the administration of PCL-F127/W379+PVP/W379 MN with the same peptide dose showed a significant reduction (2-3 Log) in CFU counting (FIG. 20A). This result indicates that the dissolvable microneedle arrays were critical in the delivery of engineered peptides to the inside of biofilm due to their direction physical penetration. To completely get rid of biofilms without debridement, the dressings was changed every 24 hours within 72 hours. The MRSA biofilms were eradicated after 3 changes of Janus-type antimicrobial dressings (FIG. 20B). In contrast, there were still about 10⁴⁻⁵ CFU/g bacteria remaining on the wounds treated by peptide-loaded nanofiber membranes alone without and with incorporation of aqueous peptides even after 3 changes (FIG. 20B). This result further confirmed the importance of microneedles for effective delivery of engineered peptides to treat biofilms. To enhance the anti-biofilm efficacy, the peptide loading in the microneedle arrays was increased from 25 mg/g to 50 mg/g. However, the increased dose showed the similar anti-biofilm efficacy, indicating that doubling the peptide loading resulted in marginal enhancement in the bacteria killing in biofilms. The density of microneedles on the surface was also increased from 100 to 150 microneedles per nanofiber membrane without changing the dose (25 mg/g). Interestingly, the bacteria were not detected after changing the Janus-type antimicrobial dressing twice and three times (FIG. 20D). Similarly, there were still about 10⁴⁻⁵ CFU/g bacteria remaining on the wounds treated by peptide-loaded nanofiber membranes alone without and with incorporation of aqueous peptides even after 2 and 3 changes (FIG. 20D). This result indicates reducing the distance between the adjacent microneedles can promote the anti-biofilm efficacy, which was likely due to the increase of the peptide diffusion area.

To further evaluate the anti-biofilm efficacy, MRSA biofilms were established in type II diabetic mice wounds (FIG. 21). Briefly, wounds were created and fixed with a splint. Ten μl of 10⁸ CFU/ml MRSA was inoculated to each wound for 24 hours and 48 hours and followed by 24 hour treatment of 2% mupirocin ointment to remove the planktonic bacteria on the wounds. The CFU of MRSA biofilms (10¹⁰-10¹² CFU/g) formed after 24 hours and 48 hours of bacteria inoculation and removal of planktonic bacteria was quantified (FIG. 21B). The biofilm formation was confirmed by LIVE/DEAD® staining and SEM observation for the tissue collected from wounds after 24 hours of MRSA inoculation and subsequent 24 hour 2% mupirocin treatment (FIGS. 21C and 21D). For in vivo anti-biofilm testing, Janus-type dressings with and without loading engineered peptides and peptides-loaded nanofiber membranes plus aqueous peptides were applied to the biofilm-containing wounds. After 3 changes (1 change per 24 hours), there were 10¹¹ CFU/g in the tissues collected from the wounds treated by Janus-type dressings without peptide loading. The treatment with F127/379-PCL core-shell nanofiber membranes plus aqueous peptides resulted in 6 Log reduction of CFU/g. Very importantly, consistent with the ex vivo results, no bacteria were detected on the wounds treated by Janus-type dressings consisting of F127/379-PCL core-shell nanofiber membranes and W379 loaded PVP microneedle arrays (FIG. 22). This result indicates the Janus-type antimicrobial dressings were also effective against biofilms in vivo.

To further understand the results, the diffusion length of peptides in the biofilm was estimated using the following equation where L_{D} is the diffusion length, D is the diffusion coefficient, and t is the diffusion time: L_{D}=√4Dt. The molecular weight of the engineered peptide used in this study is 1330 g/mol. The diffusion coefficient of peptides (D=3.1×10⁻¹² m²/s) was used to estimate the diffusion length (Lawrence, et al. (1994) Appl. Environ. Microbiol., 60(4):1166-1173). LD was estimated to be around 210 μm when t=24 hours. For the microneedle arrays, the diameter of the needle bottom is 300 μm, the diameter of the needle tip is 5 μm, and the gap distances between the adjacent microneedles are 300 or 540 μm (low density) and 150 or 330 μm (high density). Based on these estimations, the administration strategy of the Janus-type antimicrobial dressing was changed such as extending the time for the dressing change to enhance the anti-biofilm efficacy. Instead of using molecularly engineered peptides, antimicrobial agents were incorporated including silver nitrate and vancomycin to the Janus-type dressings. These dressings were tested in an ex vivo biofilm-containing human skin wound model. FIG. 23 shows that silver and vancomycin-loaded Janus-type dressings can effectively treat biofilms in human skin wounds ex vivo after changing the dressing three times by extending the time for dressing administration from 24 hours to 36 hours (FIG. 23). In addition, the dressing with high density of microneedles can further promote the antibiofilm efficacy, showing the CFU counting was not detectable after changing the dressing twice (FIGS. 23C and 23F). These results were very encouraging as the antimicrobial agents used in the dressings are currently clinically used and such dressing could be easily translated into clinics for biofilm management in chronic wounds.

The Janus-type antimicrobial dressings may be modified (e.g., by geometric design, pattern, density, composition, and loading of engineered peptides of microneedles) to further promote their anti-biofilm efficacy. In addition, the fabrication of 3D objects consisting of hierarchically assembled nanofibers with controlled alignment can be used. The 3D objects can promote cellular infiltration to form 3D tissue constructs in vitro and in vivo. Such 3D objects can also be combined with microneedle arrays to form Janus-type antimicrobial dressings that can not only eradicate biofilms but also promote wound healing. In addition, antimicrobial agents (e.g., antibiotics, silver nanoparticles/ions, gallium ions, molecularly engineered peptides) can be incorporated into the Janus-type antimicrobial dressings to have a synergistic efficacy against biofilms.

In summary, novel Janus-type antimicrobial dressings have been provided for effective treatment of biofilms in excisional wounds in human skin explants and type II diabetic wounds without debridement. The technology provides an effective intervention with great potential that can effectively treat biofilms, in particular, multi-drug resistant bacteria formed biofilms, improve quality of wound care, decrease costs, avoid amputations, and most importantly save lives of patients.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. A nanofiber structure comprising electrospun nanofibers having a core and shell morphology, wherein said nanofibers comprise an amphiphilic block copolymer, a hydrophobic polymer, and an antimicrobial.

2. The nanofiber structure of claim 1, wherein said antimicrobial is contained within the core of said nanofibers.

3. The nanofiber structure of claim 1, wherein said hydrophobic polymer is contained within the shell of said nanofibers.

4. The nanofiber structure of claim 1, wherein said hydrophobic polymer is polycaprolactone (PCL).

5. The nanofiber structure of claim 1, wherein said nanofiber structure comprises a poloxamer.

6. The nanofiber structure of claim 5, wherein said poloxamer is poloxamer 407.

7. The nanofiber structure of claim 1, wherein said nanofiber structure comprises a plurality of uniaxially-aligned nanofibers, random nanofibers, and/or entangled nanofibers.

8. The nanofiber structure of claim 1, wherein said antimicrobial is an antibiotic.

9. The nanofiber structure of claim 1, wherein said antimicrobial is a silver containing compound.

10. The nanofiber structure of claim 1, wherein said antimicrobial is an antifungal compound.

11. The nanofiber structure of claim 1, wherein said antimicrobial is an antimicrobial peptide.

12. The nanofiber structure of claim 1, further comprising a coating on said nanofibers.

13. The nanofiber structure of claim 1, wherein said coating comprises an antimicrobial.

14. The nanofiber structure of claim 12, wherein said coating comprises said antimicrobial and said amphiphilic block copolymer.

15. The nanofiber structure of claim 1, further comprising another drug or therapeutic agent.

16. The nanofiber structure of claim 15, wherein said drug or therapeutic agent is selected from the group consisting of a therapeutic agent, a growth factor, a signaling molecule, a cytokine, a hemostatic agent, an antimicrobial, and an antibiotic.

17. The nanofiber structure of claim 1, further comprising a plurality of microneedles attached or fused to said nanofiber structure or a microneedle structure attached or fused to said nanofiber structure, wherein said microneedle structure comprises a plurality of microneedles.

18. The nanofiber structure of claim 17, wherein said microneedles or microneedle structure comprises an antimicrobial and a biodegradable polymer.

19. The nanofiber structure of claim 18, wherein said biodegradable polymer is polyvinylpyrrolidone (PVP).

20. The nanofiber structure of claim 17, wherein said microneedles have an average height of less than 500 μm.

21. The nanofiber structure of claim 17, wherein the distance between microneedles in the microneedle structure is less than 300 μm.

22. A method for producing a nanofiber structure of claim 1, said method comprising co-axially electrospinning said nanofibers.

23. A wound dressing comprising a nanofiber structure of claim 1.

24. A method of treating and/or inhibiting a wound or infection in a subject, said method comprising administering a nanofiber structure of claim 1 to a wound or infection in said subject.

25. The method of claim 24, wherein said nanofiber structure is in a wound dressing.

26. The method of claim 24, wherein said infection is a bacterial infection.

27. The method of claim 24, wherein said infection is a bacterial biofilm infection.

28. The method of claim 24, wherein said infection is a fungal infection.
